(19) United States
(12) Patent Application Publication
(10) Pub. No.: US 2008/0057491 A1
(43) Pub. Date: Mar. 6, 2008

(54) Substrates and inhibitors of antiplasmin cleaving enzyme and methods of use

(76) Inventors: Patrick A. McKee, Oklahoma City, OK (US); Kyung N. Lee, Oklahoma City, OK (US); Kenneth W. Jackson, Edmond, OK (US); Victoria J. Christiansen, Oklahoma City, OK (US)

Correspondence Address: DUNLAP CODDING & ROGERS, P.C., PO BOX 16370, OKLAHOMA CITY, OK, 73113, US

(21) Appl. No.: 11/811,002

(22) Filed: Jun. 6, 2007

Related U.S. Application Data
(63) Continuation-in-part of application No. 10/774,242, filed on Feb. 6, 2004, now Pat. No. 7,309,774.
(60) Provisional application No. 60/445,774, filed on Feb. 7, 2003.
(60) Provisional application No. 60/811,568, filed on Jun. 7, 2006.
(60) Provisional application No. 60/836,365, filed on Aug. 8, 2006.

(51) Int. Cl. C12Q 1/00; C07K 14/00; C07K 5/00; C07K 7/06; C07K 7/08

(52) U.S. Cl. 435/4; 530/324; 530/325; 530/326; 530/327; 530/328; 530/330

(57) ABSTRACT

The present invention is directed toward inhibitors of antiplasmin cleaving enzyme for use in various therapies related to fibrin and α₂-antiplasmin, and to substrates of APCE, which may be used, for example, in screening methods for identifying such inhibitors. The present invention is further directed to methods of treating or inhibiting atherosclerosis and thrombus disorders by altering the rations of types of plasma α₂-antiplasmin.

### CROSS-REFERENCE TO RELATED APPLICATIONS

The present application is a continuation-in-part of U.S. Ser. No. 10/774,242, filed Feb. 6, 2004, which claims the benefit under 35 U.S.C. 119(e) of U.S. Provisional Application Ser. No. 60/445,774, filed Feb. 7, 2003. The present application also claims the benefit under 35 U.S.C. 119(e) of U.S. Provisional application Ser. No. 60/811,568, filed Jun. 7, 2006 and U.S. Provisional Application Ser. No. 60/836,365, filed Aug. 8, 2006. The entire contents of each of the applications listed above is hereby expressly incorporated herein by reference in its entirety.

### STATEMENT REGARDING FEDERALLY SPONSORED RESEARCH OR DEVELOPMENT

Some aspects of this invention were made in the course of Grant M01-RR14467 awarded by the National Center for Research Resources-National Institutes of Health and therefore the Government has certain rights in some aspects of this invention.

### BACKGROUND

The present invention is related to, but not limited to, substrates and inhibitors of α₂-antiplasmin cleaving enzyme and to screening methods for identifying such inhibitors, and to methods for treating conditions involving fibrin and α₂-antiplasmin, including plaque and clot formation and atherosclerosis.

α₂-Antiplasmin (α₂AP) is a blood plasma protein that rapidly and specifically inhibits the enzyme, plasmin, which digests blood clots, whether presenting early as intravascular platelet-fibrin deposits or as partially or completely occlusive thrombi. Similarly, plasmin and α₂AP activities are important to the development and survival of fibrin as occurs in inflammation, wound healing and virtually all forms of cancer and its metastases.

Human α₂-antiplasmin (α₂AP), also known as α₂-plasmin inhibitor, is the main inhibitor of plasmin. Plasmin plays a critical role in fibrin proteolysis and tissue remodeling. The physiologic relevance of plasmin inhibition by α₂AP to blood clotting and fibrinolytic homeostasis is supported by the following observations: (1) the rate of free plasmin inactivation by circulating α₂AP is much faster than fibrin(ogen) digestion by plasmin, thereby eliminating the possibility of a systemic lytic state and consequent bleeding; (2) α₂AP is cross-linked to forming fibrin by activated blood clotting factor XIII (FXIIIa) and inhibits plasmin-mediated lysis in direct proportion to the amount incorporated; and (3) patients with homozygous α₂AP deficiency manifest serious hemorrhagic tendencies, while heterozygotes tend to bleed only after major trauma or surgery. Human α₂AP is synthesized primarily in the liver, and during circulation in plasma, the secreted precursive form, Met-α₂-antiplasmin (Met-α₂AP), a 464-residue protein having methionine as the N-terminus, undergoes proteolytic cleavage between Pro12 and Asn13 (the P₁—P₁′ scissile bond) to yield Asn-α₂-antiplasmin (Asn-α₂AP), a 452-residue version with asparagine as the N-terminus. Met-α₂AP accounts for approximately 30% of circulating α₂AP, and Asn-α₂AP accounts for approximately 70%.

When the Met-form of α₂AP was found in plasma and its gene sequenced, there initially appeared to be a discrepancy in one of the nucleotides encoding the sixth amino acid. Two groups found a cytidine (C) resulting in Arg as the sixth amino acid, and one group found thymidine (T), resulting in Trp at that position. It was suggested that the difference was due to one group having used liver carcinoma cells as a source of DNA, while the other two groups used normal cells. It has now been determined that both Arg6 and Trp6 forms of Met-α₂AP exist in normal human plasma samples. An investigation of a mutant α₂AP in a family with bleeding tendencies identified the mutation responsible for the ineffective α₂AP along with three polymorphisms in the α₂AP gene including this C/T single nucleotide polymorphism (SNP); this study examined 30 normal blood donors and reported an allelic frequency of 0.81/0.19 for the C/T SNP. No larger studies of a normal population have been done to examine the frequency of homozygotes and heterozygotes, or whether genotype might affect ratios of Met- to Asn-α₂AP in plasma. The Arg6Trp SNP was apparently assumed to be a silent polymorphism, but biochemical examination of the two polymorphic forms of Met-α₂AP on yielding the derivative form, Asn-α₂AP, its incorporation into fibrin and the impact on fibrinolysis have never been assessed prior to the present work.

### SUMMARY OF THE INVENTION

The present invention is directed toward inhibitors of antiplasmin cleaving enzyme for use in various therapies related to fibrin and α₂-antiplasmin, and to substrates of APCE, which may be used, for example, in screening methods for identifying such inhibitors. The present invention is further directed to, but not limited to, methods of treating or inhibiting atherosclerosis and thrombus disorders by altering the ratios of types of plasma α₂-antiplasmin and inhibiting APCE.

### BRIEF DESCRIPTION OF THE DRAWINGS

FIG. 1. Met-α₂AP as percent of total α₂AP and by genotype. α₂AP was purified from each plasma of persons with RR (n=15), RW (n=15) and WW (n=5) genotypes and amino-terminal sequences determined by Edman degradation. Percent Met-α₂AP was calculated from picomole recoveries of Met and Asn in the first cycle.

FIG. 2. Plasma APCE levels in a normal population and partitioned by genotype. APCE levels in plasma samples were determined by ELISA. Panel A. Histogram of APCE concentrations in a normal population (n=109). Panel B. APCE levels by Met-α₂AP genotype.

FIG. 3. APCE cleavage of polymorphic forms of Met-α₂AP. Equal amounts (40 μg) of purified Met-α₂AP(Arg6) and Met-α₂AP(Trp6) were digested by APCE. After stopping the reaction at selected times, samples were assessed by electrospray mass spectrometry for the quantity of the amino-terminal 12-amino acid peptide produced from each Met-α₂AP form.

FIG. 4. Effect of Met-α₂AP genotype on generation of Asn-α₂AP in plasma with time. A plasma sample from a person of the RR, RW or WW genotype was incubated at 29° C.; at selected times α₂AP was purified from each sample and subjected to amino-terminal sequence analysis. The ratio of Asn-α₂AP/Met-α₂AP was calculated from picomole recoveries of Met and Asn in the first cycle.

FIG. 5. Plasma clot lysis times (PCLT) by Met-α₂AP genotype. PCLTs were determined on plasma samples from RR, RW and WW persons. Panel A. PCLT values were divided by genotype and plotted as mean ±S.E.M. for the total population, men only and women only. Panel B. Percentage of plasmas that did not lyse (n=14) compared to percentage of total population (n=200) within each genotype.

FIG. 6. Effect of APCE removal on conversion of Met-α₂-AP to Asn-α₂-AP with time. Plasma was drawn from a person of RR Met-α₂AP genotype and divided into three aliquots. One aliquot was mixed with an APCE F19 mAb (F19) bound to chromatography beads and incubated at 4° C. to remove APCE. The second aliquot was incubated at 4° C. with a non-specific rabbit a-goat Ab (RAG) bound to beads. The third aliquot (RR) received no treatment. After removal of beads, each sample was incubated at 29° C. and Asn-α₂AP/Met-α₂AP ratios determined at selected times. In addition, F19-bound beads and RAG-bound beads were boiled with SDS to remove antibody-bound protein. Samples were electrophoresed on 10% Bis-Tris SDS-PAGE gels and blotted to nitrocellulose. APCE (97 kDa) was identified by Western blotting using a goat Ab to its amino-terminal region, and visualized with a chemiluminescent substrate.

FIG. 7 shows the inhibitor effect of Val-boroPro on conversion of Met-α₂AP to Asn-α₂AP by APCE.

FIG. 8 shows a non-limiting exemplary sequence of the FRET peptide (SEQ ID NO:5).

FIG. 9 shows various substitutions which could be made in the P₁′ position of the P₂—P₁—P₁′ group, and various substitutions which could be made to residues upstream (N-terminal direction) of the P₂ residue, and to the residues downstream (C-terminal direction) of the P₁′ residue (SEQ ID NO:6).

FIG. 10. LC/MS analysis of antiplasmin peptides (SEQ ID NO:7) for substrate preference determination in position P₇ (sixth amino acid upstream of the P₁ pro) Those with positively charged His(H), Lys(K) and Arg (R) enhanced the cleavage rate of the Pro₁₂-N₁₃ bond.

FIG. 11. Importance of the position of Arg relative to the Pro-Asn scissile bond. Four similar peptides based upon the antiplasmin amino-terminal sequence (4-17) were digested by APCE and then LC/MS analysis was performed. Each reaction solution contained one experimental peptide and the control peptide, with Arg in the P₈, P₇, P₆, P₅ or P₄ (not shown) position and glycines in the other three variable positions for any given peptide. Substitution at P₄ gave a result that was about the same as P₈.

FIG. 12. LC/MS analysis of antiplasmin peptides for substrate preference determination in positions P₄, P₃, P₂, P₁. Peptide libraries representing positions six through 17 of the human antiplasmin sequence were treated with APCE (also known as soluble FAP). The phenylalanine in red was added to enhance binding to the reversed-phase HPLC column. The amino acids common to proteins, less cysteine were substituted one at a time in each of the positions P₁ through P₄ (SEQ ID NO:8). The relative rates of cleavage of the Pro-Asn bond were determine by LC/MS analysis

FIG. 13 shows an alternative embodiment of an amino acid sequence (SEQ ID NO:9) comprising a portion of a FRET peptide or inhibitor of the present invention. The sequence could be extended in the C-terminal direction for example by the sequences shown in FIG. 9 extending from N₁₃, in the N-terminal direction or extending from N₁₃ in the C-terminal direction.

FIG. 14 shows the APCE inhibitory effect of various inhibitory peptidomimetics (SEQ ID NO: 11-13) at inhibitor concentrations of 20 μM, where the proline has been substituted with pipecolic acid.

### DETAILED DESCRIPTION OF THE INVENTION

Human Met-α₂AP is a physiologically important substrate of APCE, since proteolytic cleavage between Pro12-Asn13 yields the more active derivative, Asn-α₂AP, which becomes cross-linked significantly faster to fibrin by FXIIIa than Met-α₂AP, and, as a consequence, enhances the resistance of fibrin to digestion by plasmin. Since human APCE augments the inhibition of fibrinolysis by increasing the availability of the faster cross-linking form (i.e., Asn-α₂AP), potent and selective inhibitors of APCE which have been discovered as described herein allow titration of Asn-α₂AP production to lower in vivo levels, and thereby enhance both endogenous and exogenous fibrinolysis (fibrin removal). Bleeding complications using the treatments described herein are unlikely, based on the observation that persons heterozygous for α₂AP deficiency have minimal hemorrhagic risk. Hence, a window of safety exists for lowering α₂AP function in healthy persons. Particularly in clinical situations where fibrin formation is likely, APCE inhibitors will result in decreased amount of Asn-α₂AP available for cross-linking to fibrin as thrombi develop or inflammation progresses. Then endogenous levels of generated plasmin, or plasmin produced by administering small amounts of a plasminogen activator, might be sufficient to effect fibrin removal so that vessel patency and organ function are maintained and bleeding risk is minimized. As noted above, APCE cleaves Met α₂AP to the derivative Asn α₂AP, which is more efficiently incorporated into fibrin and consequently makes it strikingly resistant to plasmin digestion. APCE thus represents a new target for pharmacologic modulation and inhibition of the fibrinolytic system, since less generation and therefore less incorporation of Asn-α₂AP results in a more rapid removal of fibrin by plasmin during atherogenesis, thrombosis, and inflammatory states.

The present invention is directed toward inhibitors of antiplasmin cleaving enzyme for use in various therapies related to fibrin and α₂-antiplasmin, and to substrates of APCE, which may be used, for example, in screening methods for identifying such inhibitors. The present invention is further directed to, but not limited to, methods of treating or inhibiting atherosclerosis and thrombus disorders by altering the ratios of types of plasma α₂-antiplasmin. In a preferred embodiment, inhibition of APCE is defined herein as at least 50% inhibition of activity of APCE, for example, at an inhibitor concentration of 20 μM.

Asn-α₂AP crosslinks to fibrin at a rate of about 13-fold faster than Met-α₂AP. A faster crosslink rate results in a greater number of antiplasmin molecules bound to newly formed fibrin and a resultant enhanced resistance to fibrinolysis. Inhibition of plasma APCE can thus decrease the number of antiplasmin molecules crosslinked thereby resulting in clots that are more easily removed during fibrinolysis. Therefore an inhibitor specific for APCE can be used to regulate fibrinolysis. As noted above, human Met-α₂AP exists in two polymorphic forms at position six in the mature sequence, with arginine predominant and tryptophan accounting for a lesser percentage. It has been discovered herein that inhibition of APCE can alter the α₂AP ratio from approximately 30% Met-α₂AP:70% Asn-α₂P to approximately 60%-70% Met-α₂AP:40%-30% Asn-α₂AP in the plasma.

### Modulation of α₂-Antiplasmin Ratio

In the present work, we determined the prevalence of the polymorphism in a much larger normal population and then assessed whether it relates to the inhibitory function of α₂AP. Provided herein are results regarding (1) genotype frequencies of the Arg6Trp SNP in Met-α₂AP; (2) how each form affects susceptibility to cleavage by APCE; (3) the percent of Met-α₂AP in plasma for each of the two polymorphisms; (4) plasma clot lysis times in relation to genotype; and (5) evidence that removal of circulating APCE prevents conversion of Met- to Asn-α₂AP.

Materials and Methods

Materials

Fresh frozen human plasma for the purification of proteins was purchased from the Sylvan Goldman Blood Institute (Oklahoma City, Okla.). Hybridoma cells secreting the F19 antibody were purchased from American Type Culture Collection (ATCC) (Manassas, Va.) and grown in serum-free media; the F19 antibody was purified from culture media using MEP-Hypercel chromatography (Pall, East Hills, N.Y.). Institutional review board (IRB) approval was obtained from University of Oklahoma Health Sciences Center for these studies (IRB #10142 and 12189).

Isolation of α₂AP

Mixtures of Met-α₂AP and Asn-α₂AP were isolated by a modification of a published purification procedure using plasminogen kringles 1-3 attached to Sepharose 4B as an affinity matrix. Met-α₂AP and Asn-α₂AP were separated by immunoaffinity chromatography as previously described. The ratio of Met-α₂AP to Asn-α₂AP in plasma samples was determined by comparison of picomole recovery of Met versus Asn in cycle one during automated protein sequencing by Edman degradation (Applied Biosystems Procise model 492, Foster City, Calif.).

Isolation of APCE

APCE was purified from human plasma as previously described (U.S. Ser. No. 10/774,242). Briefly, a combination of ammonium sulfate precipitation, hydrophobic interaction, and immunoaffinity chromatography were used for purification. Before storing at −80° C., glycerol was added to the pure APCE to give a final concentration of 20%.

Determination of α₂AP Genotype

Two hundred randomly responding normal volunteers, self-reported as healthy and free of acute illness, were recruited to donate blood for determination of genotype frequency and plasma clot lysis time (PCLT; see next section) in a normal population. DNA was isolated from whole blood of each donor using the AquaPure Genomic DNA Blood Kit (Bio-Rad, Hercules, Calif.). The portion of DNA encompassing the Arg6Trp SNP was amplified by polymerase chain reaction using oligonucleotide primers (5′-GACCTCCTATCCTCATCCCTTT (SEQ ID NO:1) and 5′-CTGGTTCGGCCCGCTAGTTAG (SEQ ID NO:2)), dNTPs (Takara Mirus Bio, Madison, Wis.), and Platinum Taq DNA polymerase (Invitrogen, Carlsbad, Calif.). Following amplification, the PCR product was purified, using the MinElute PCR Purification Kit (Qiagen Operon, Alameda, Calif.) and sequenced, using an ABI3730 automated DNA sequencer.

Measurement of Plasma Clot Lysis Time

To measure plasma clot lysis time (PCLT), a mixture of 1 unit/ml thrombin, 16 mM CaCl₂ and 45 IU/ml urokinase (uPA) (Abbott, Chicago, Ill.) was added to each volunteer's plasma to catalyze essentially instant fibrin clot formation and to initiate fibrinolysis; the rate of plasma clot lysis was determined by a turbidimetric microtiter plate method.

Determination of Cleavage Rates

Reaction mixtures containing equal amounts (40 ug) of pure Met-α₂AP(Arg6) and pure Met-α₂AP(Trp6) were digested by APCE. At selected times the digestion was stopped by decreasing the pH from 7.5 to 4.0 with trifluoroacetic acid. Proteins were removed from the mixture by Microcon (Millipore, Bedford, Mass.) centrifugal ultrafiltration using a 30 kDa cutoff membrane. The peptides were isolated from the ultrafiltered digestion mixture by binding to POROS-50 reversed-phase media (Applied Biosystems, Foster City, Calif.) packed into a glass purification capillary (Proxeon, Odense, Denmark). The peptides were then eluted from the POROS-50 directly into a metal coated glass nanospray capillary with 2.0 μl of 0.5% acetic acid in 1:1 methanol/water. The nanospray capillary was mounted on the nanospray ionization source of a QSTAR ESI-Quad-TOF mass spectrometer operated under Analyst QS software version 1.0 (Applied Biosystems, Foster City, Calif.) with an ionspray voltage of 1400 volts. Data was collected over a mass range of 300 to 1500 Da. The relative quantities of the two α₂AP amino-terminal 12-amino acid peptides produced, MEPLGRQLTSGP (SEQ ID NO:3), Mr=1284.65 for Met-α₂AP(Arg6) and MEPLGWQLTSGP (SEQ ID NO:4), Mr=1314.63 for Met-α₂AP(Trp6), were determined by summing the areas of the four most abundant isotope peaks for the observed charge forms for each peptide. The data for each time point was normalized by a similar quantification of an added inert internal standard peptide that contained no proline.

Determination of APCE Antigen Level

An enzyme-linked immunosorbant assay (ELISA) was developed to determine antigen levels in human plasma. A goat antibody to the amino-terminal 15-amino acid sequence of APCE was prepared, using as the immunogen a multiple antigenic peptide (MAP) constructed in our laboratory to contain eight copies of the amino-terminal peptide linked via their carboxyl-termini to a core peptide of seven lysines. This goat MAP (amino-terminal 15 residue APCE peptide) antibody was bound to white high-binding polystyrene assay plates (Corning, Corning, N.Y.) and used as the capture antibody. After incubation with dilutions of plasma, a monoclonal antibody purified from commercially available F19 hybridomas (ATCC, Manassas, Va.) was applied, followed by peroxidase conjugated anti-mouse antibody (Sigma, St. Louis, Mo.). A chemiluminescent substrate, SuperSignal ELISA Pico (Pierce, Rockford, Ill.), was added and luminescence was monitored using a BIO-TEK FL600 plate reader (Winooski, Vt.). Antigen level was quantitated using purified human APCE as the standard.

Removal of APCE from Plasma

The F19 mAb was linked to POROS EP 20 poly(styrenedivinylbenzene) perfusion chromatography beads (Applied Biosystems, Foster City, Calif.) and a nonspecific antibody, rabbit anti-goat, was linked to the same type of media. Plasma from a single donor of the RR genotype was divided into 3 aliquots, diluted 1:1 with PBS and incubated separately with each of the two bead-linked antibodies, non-specific Ab or the F19 mAb, overnight at 4° C. The third aliquot received no treatment. The beads were removed from the plasma by filtration and the plasma was then incubated at 29° C. Aliquots were removed at zero time, 24 hours and 48 hours. α₂AP was purified from each aliquot and the Asn-α₂AP/Met-α₂AP ratio was determined as previously described. After removal from the plasma, the F19 mAb beads were washed with 25 mM Na PO₄/0.5M NaCl and then boiled with SDS loading buffer. The SDS buffer extract was then separated by electrophoresis on a 100% Bis-Tris gel (Invitrogen, Carlsbad, Calif.) and blotted to nitrocellulose. APCE was identified by Western blotting using the goat amino-terminal MAP antibody as described previously in this Methods section and visualized using SuperSignal West Femto Maximum Sensitivity Chemiluminescent Substrate (Pierce Biotechnology, Inc., Rockford, Ill.).

Results

α₂AP Genotype Determination

A group of 201 normal volunteers, who were recruited in two sets of approximately 100 people each, separated by about two years, provided blood samples for determining the α₂AP C/T SNP frequency. The total population consisted of 61 men and 139 women 21 to 69 years of age with an ethnicity that closely matched the demographics for an Oklahoma population as listed for the year 2000 on the U.S. Census web site (factfinder.census.gov). Genotype was determined for 200 of the subjects. Only one DNA sample did not amplify by polymerase chain reaction, possibly due to a mutation that prevented binding of one of the primers, although this has not been further explored. The genotype frequencies for the two normal populations were essentially the same with less than 1% difference for any of the genotypes. After combining the two sets, the frequencies for the entire population were RR 62.5%; RW 34.0%; WW 3.5%. The R allele had a frequency of 79.5% and the W allele had a frequency of 20.5%. There was no difference in the genotype frequency between men and women. Because the population was 72% Caucasian, with the other 28% split between 6 ethnic categories, it was not possible to determine whether genotype varied among ethnic groups.

Met-α₂AP and Asn-α₂AP Levels in Plasma

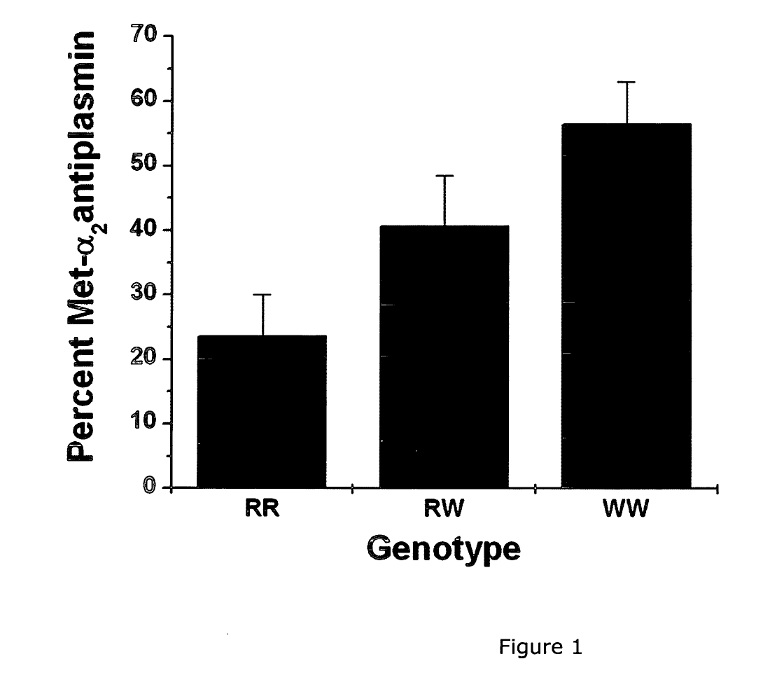

α₂AP was purified from the plasmas of 15 persons of the RR genotype; 15 persons with RW; and 5 with WW genotype. The α₂AP was sequenced by Edman degradation and picomole recoveries of Met and Asn were determined for the first cycle to calculate the percent of Met-α₂AP and Asn-α₂AP. FIG. 1 shows significantly different (p<0.001) results that partitioned by genotype with WW having the highest percentage of Met-α₂AP (56.4%); RR, the least (23.6%), and RW falling in between, at (40.6%).

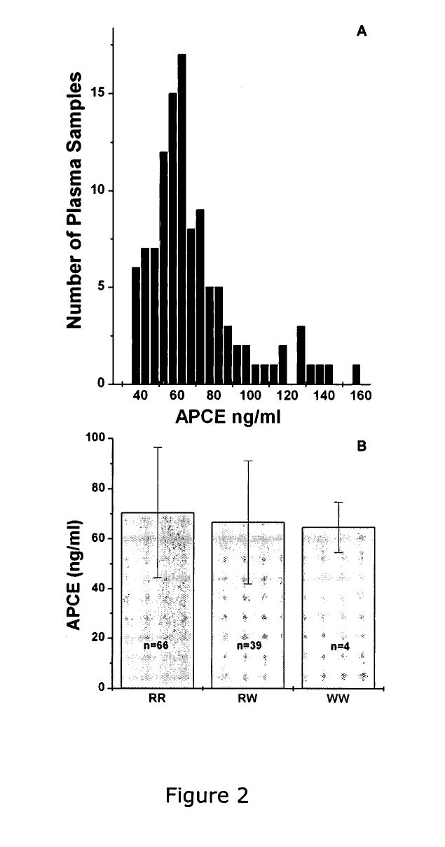

To understand the mechanism for the varying percentages of Met-α₂AP in human plasma, we examined whether a variation in the level of enzyme that converts Met-α₂AP to Asn-α₂AP, i.e., APCE, might explain the differences among the genotypes. To investigate this possibility, we developed an ELISA method for quantitating APCE antigen level in plasma and then determined APCE concentrations in plasma from 109 subjects in our normal population. As seen in FIG. 2a, there is a distribution of APCE levels in normal human plasma, ranging from 38 to 159 ng/ml that did not correlate with age or gender, and despite the suggestion of a possible association of APCE levels with genotype (RR: 70.4±26; RW: 66.6±24.5; WW: 64.7±10.1), the differences were not statistically significant (FIG. 2b). Therefore APCE levels do not appear to account for the variation of Met-α₂AP levels among genotypes.

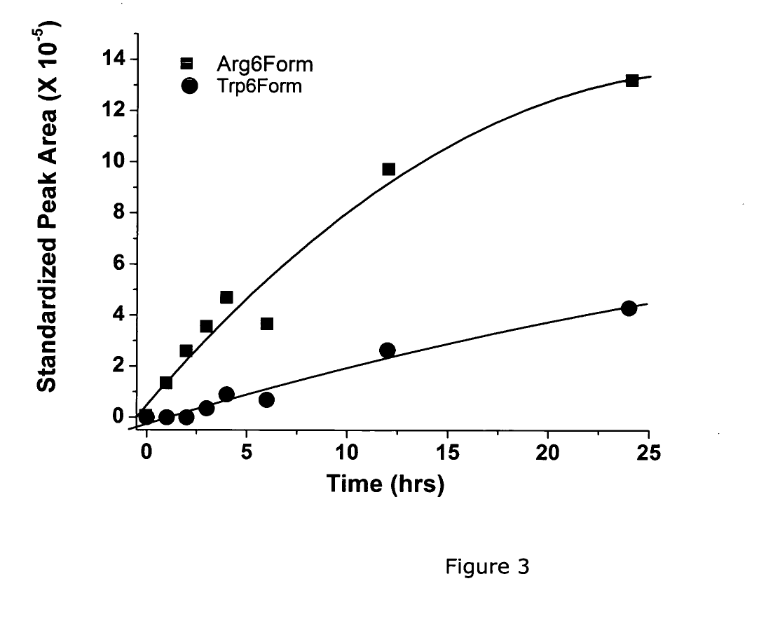

Another explanation for the different Met-α₂AP percentages in the three genotypes that was explored was that Met-α₂AP(Arg6) is a better substrate for APCE than Met-α₂AP(Trp6). To test this hypothesis, α₂AP was purified from RR and WW plasma and the cleavage rate of each polymorphic form, Met-α₂AP(Arg6) and Met-α₂AP(Trp6), was determined, using mass spectrometry to monitor the generation of the 12-residue amino-terminal peptide with time. As seen in FIG. 3, comparisons of reaction rates were based on linear regression analysis of early time points and showed that APCE cleaves Met-α₂AP(Arg6) approximately 8-fold faster than Met-α₂AP(Trp6).

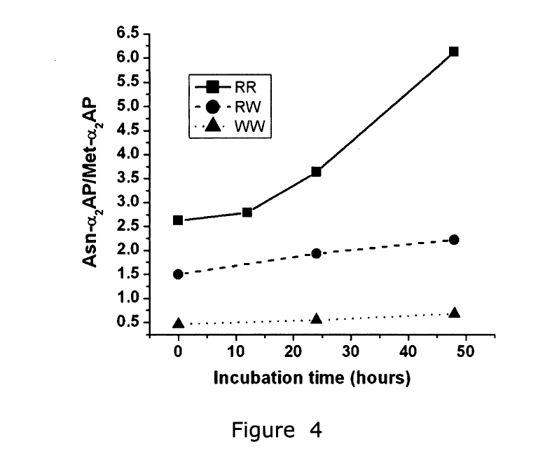

To examine whether the cleavage rate of Met-α₂AP in whole plasma gave similar results based on the presence of R or W in position 6, fresh plasma samples from individuals whose genotype was determined to be RR, RW and WW were obtained, incubated at 29° C., with each being analyzed at 0, 24 and 48 hours for Asn-α₂AP/Met-α₂AP ratios. α₂AP was purified from each sample and ratio of the two polymorphic forms was determined by picomole recovery of the amino-terminal residue in the first cycle of Edman degradation. As seen in FIG. 4, Asn-α₂AP increased at a much greater rate in plasma from a normal volunteer of the RR genotype than in either the RW or WW genotype patients. These results obviously concur with those from reaction mixtures of pure α₂AP and APCE that showed APCE cleaves Met-α₂AP(Arg6) approximately 8-fold faster than Met-α₂AP(Trp6).

Plasma Clot Lysis

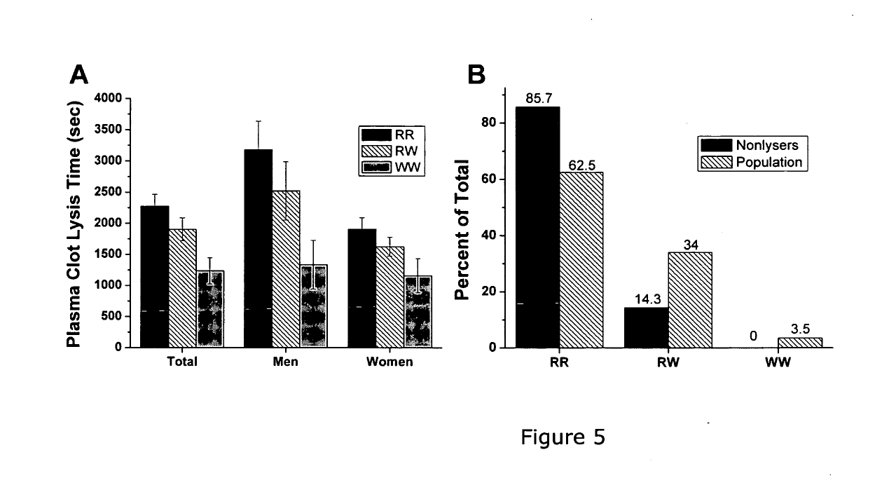

Plasma clot lysis times (PCLTs) were determined on blood samples from the 200 women and men comprising the normal population. We did not attempt to control for variations of clot lysis that are known to occur due to activator release, activator-inhibitor interactions, fibrinogen level or to effects of lipids on enzyme-substrate mechanisms; hence, gender, age, adrenergic status, smoking, obesity, alcohol consumption, serum lipid and fibrinogen measurements, medication, diurnal activities, etc., were disregarded as qualifiers for volunteer participation in the study. Instead, “all-comers” were entered so long as they reported themselves to be in good health, believing that study participants' lysis times should more or less reflect the “average” of all physiologic/pharmacologic effects within each genotype at any point in time and that if indeed the polymorphism detectably affected plasma clot lysis under our conditions, then its potential importance would be underscored. Important to emphasize is that mean PCLTs for men were significantly prolonged compared to values for women (p=0.0002). As depicted in FIG. 5, mean PCLTs for the RR, RW and WW genotype groups exhibited an impressive linear decrease, but the differences between mean PCLTs among the three genotypes were not statistically significant. As shown in panel A of FIG. 5, after separating genotypes by gender, the differences for the mean PCLT among genotypes showed an obvious trend toward shorter lysis times for the WW genotype in both men and women. Since the distribution of PCLTs was skewed, non-parametric statistical methods were used to analyze the data. Medians for the RR and RW were similar and the distributions overlapped, suggesting that the R allele is dominant. When RR and RW groups were merged and compared to WW, after accounting for variation due to gender, the differences approached significance (p=0.061). As noted in FIG. 5 panel B, 12 (10%) persons of the RR genotype and 2 (3%) of the RW genotype had plasma clots that remained totally intact during the entire one hour assay period; no person of WW genotype had a lysis time >2100 seconds.

Removal of APCE from Plasma

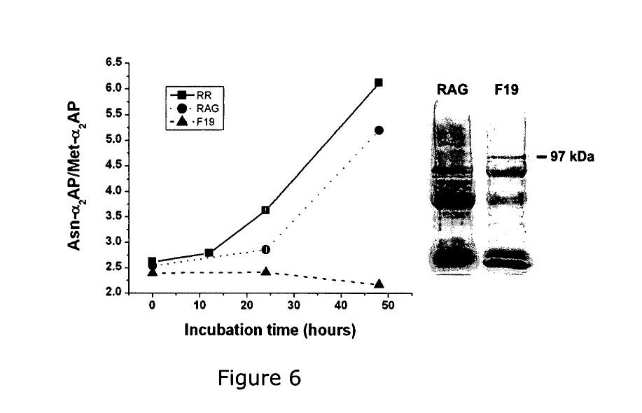

In an effort to definitively establish that APCE is the enzyme responsible for the conversion of Met-α₂AP to Asn-α₂AP, we removed APCE from plasma by incubating the plasma with the FAP-specific monoclonal antibody, F19, covalently attached to POROS chromatography beads. After removal of the beads, incubation of the plasma was continued for selected times at 29° C. to allow conversion of Met-α₂AP to Asn-α₂AP. The graph in FIG. 6 shows that the Asn-α₂AP/Met-α₂AP ratio in the control plasma, not incubated with the POROS-bound antibody, increased from 2.62 to 6.12 after 48 hours of incubation. In a second control, namely, plasma incubated with a non-specific antibody bound to POROS chromatography beads, a significant increase in the Asn-α₂AP/Met-α₂AP ratio from 2.54 to 5.2 occurred after 48 hours. However, plasma incubated with the F19 antibody, showed no increase in the Asn-α₂AP/Met-α₂AP ratio, 2.4 to 2.17, over the same incubation period, thereby indicating removal of APCE by the F19 antibody. These results clearly demonstrate that removal of APCE from plasma abrogates conversion of Met-α₂AP to the shorter, faster fibrin-crosslinking form, Asn-α₂AP. As further support for this conclusion, we also demonstrated by Western blot analysis that it was indeed APCE that was bound and removed by the bead-linked F19 mAb. Western blot analysis shown in FIG. 6 shows an APCE monomeric 97 kDa band in the sample eluted from F19-linked beads; no such band was present on the rabbit anti-goat (RAG) Ab-linked beads. Other bands visible were the result of non-specific binding of the secondary Ab to immunoglobulin in the samples, which leaches off the beads during extraction by boiling in SDS.

In the present work, the two populations of normal volunteers we analyzed in separate time frames were essentially identical, with the pooled genotype frequency being RR, 62.5%; RW, 34.0%; and WW, 3.5%. The allelic frequency values of 0.795/0.205 are in accord with the only other study of which we are aware, namely that of Lind and Thorsen, who reported values of 0.81/0.19 for the single nucleotide transition in 30 normal blood donors. Because this polymorphism occurs in the 12-residue amino-terminal peptide that is removed from the longer, precursive form of α₂AP, and given that a positively charged hydrophilic arginine (R) is substituted with a hydrophobic amino acid, tryptophan (W), we questioned whether such a difference might affect the rate of cleavage of the peptide by APCE and subsequent incorporation into forming fibrin. We found that pure APCE cleaved pure Met-α₂AP of the WW genotype about 8× slower when compared to pure Met-α₂AP from plasma of RR individuals. FIG. 4 clearly shows that the native precursive Met-α₂AP/derivative Asn-α₂AP ratios in plasma samples containing each of the two polymorphic forms of Met-α₂AP change spontaneously with time when freshly drawn plasma is allowed to incubate at 29° C. This cleavage must be due to the naturally occurring plasma levels of APCE, since as shown in FIG. 6, removal of APCE with a specific monoclonal antibody totally aborted generation of derivative Asn-α₂AP during the same incubation time. Since this cleavage occurs spontaneously within circulating blood, ratios of precursive Met-α₂AP/derivative Asn-α₂AP should vary within the circulating plasma from the three genotypes, which in fact we demonstrated by quantitative amino-terminal analysis of the precursive/derivative α₂AP forms for each of the 35 persons analyzed. As predicted, persons of the RR genotype had the least amount of circulating Met-α₂AP (23.6%); with RW intermediate, (40.6%); and WW the highest, (56.4%). Our results cannot be explained by variation in APCE levels, since as depicted in FIG. 2b, antigen levels were not significantly different among the three genotypes.

The relationship between RR, RW and WW genotypes and corresponding Met-α₂AP/Asn-α₂AP ratios raised the possibility that the latter would impact individuals' fibrinolytic activities so that over the course of one's life, vulnerability of intravascularly generated fibrin to endogenous fibrinolysis, and consequently its survival, are differentially affected by Met-α₂AP genotype. As a consequence, persons of the WW genotype would have Met-α₂AP that is less susceptible to conversion to Asn-α₂AP due to cleavage by APCE and therefore less effectively incorporated into forming fibrin, thereby making any generated fibrin more susceptible to digestion by plasmin. Experiments were conducted to demonstrate this using whole plasma to approximate native conditions as closely as possible. As indicated in the prior section, only minimal effort was made to standardize conditions under which all samples were drawn from healthy volunteers, on the basis that if indeed the WW genotype group had shortened fibrinolysis times, then odds should favor this being the case for the majority of time. Most of the perturbants known to affect fibrinolytic times—either acutely or chronically—are in play over one's lifetime, and in spite of such influences, we posited that on the average, fibrinolytic status would segregate according to Met-α₂AP genotype. Noteworthy is that in all our analyses, persons of RR genotype had the longest mean PCLT, with the RW group intermediate, and those in the WW genotype the shortest, suggesting that WW persons chronically have a more active fibrinolytic system than the RW group, and certainly greater activity than those with the RR genotype. The RR genotype contained a higher than expected percentage of persons whose fibrin never lysed, and if these were assigned PCLT values one second above the maximum measured value for any person in our study, then for men, the association of mean lysis times with RR and RW achieved significance at the p<0.05 level. Our results indicate that over one's lifetime, the W allele can serve as a “protection factor” (in contrast to the well-understood term, “risk factor”) by increasing the susceptibility of developing intravascular thrombi to removal by plasmin thereby decreasing the risk for atherosclerosis.

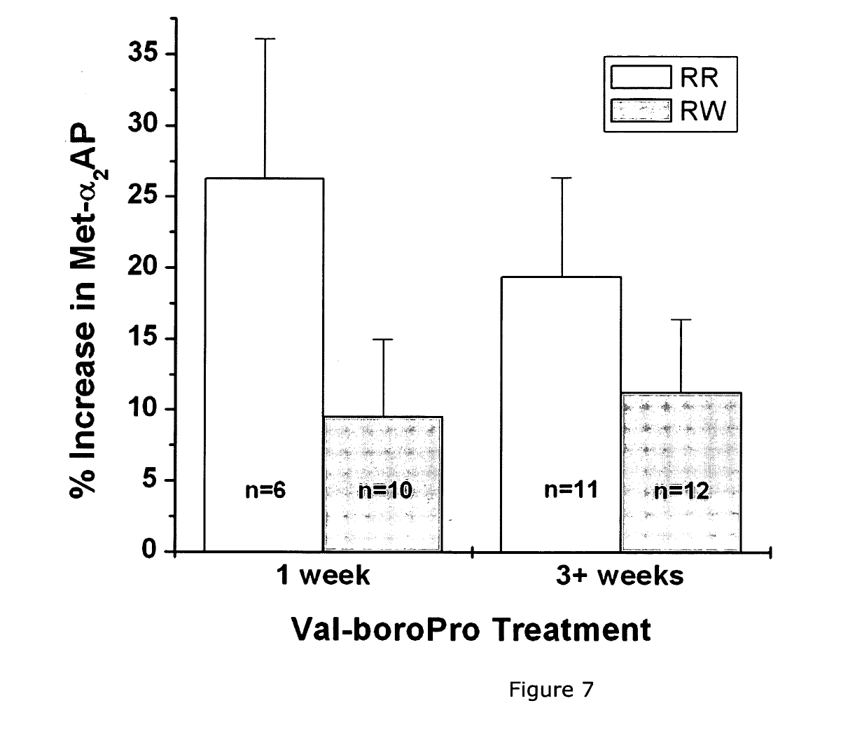

Further, these results demonstrate the utility in increasing Met-α₂AP/Asn-α₂AP ratios to approximate those that accelerate fibrinolysis. By decreasing the function of α₂AP—essentially the sole in vivo inhibitor of plasmin—to levels that carry little risk of major bleeding, as exemplified in heterozygote deficiencies of α₂AP function, and a chronic level of endogenous lytic activity sustained, then the survival and participation of intravascular fibrin-platelet thrombi in the atherosclerotic process can be reduced. In other work, plasma samples were taken from metastatic colon cancer patients who were experimentally treated with oral Val-boroPro (talabostat) which we have demonstrated is a non-specific inhibitor of APCE. FIG. 7 shows that the APCE inhibitor Val-boroPro raises the percentage of Met-α₂AP in plasma indicating less conversion of Met-α₂AP to Asn-α₂AP by APCE.

Fibrin is key to stabilization of platelets as they adhere and aggregate at a site of injury of an arterial wall during the earliest stage of plaque development. Fibrin continues to be laid down as oxidized lipid and macrophages infiltrate the site of injury, and the plaque grows to gradually encroach on the diameter of the lumen with risk of rupture and acute occlusive thrombus formation. During all these stages, if fibrin contains high or maximal α₂plasmin, then the vulnerability of the fibrin to removal by the endogenous fibrinolytic system is decreased than if the fibrin contains lesser amounts of α₂AP inhibitor. With higher circulating blood levels of precursive α₂AP, (Met-α₂AP) less total α₂AP becomes crosslinked to fibrin, causing the fibrin to be more easily digested and cleared from the plaque site by the fibrinolytic enzyme, plasmin. If, however, its derivative, Asn-α₂AP dominates, then fibrin is more resistant to removal by fibrinolysis. By inhibiting APCE, then Met-α₂AP is increased in concentration in blood and any fibrin that forms is more easily removed by one's own fibrinolytic system.

### Fluorescence Resonance Energy Transfer Peptide Substrates

The present invention in one embodiment contemplates a fluorescence resonance energy transfer peptide (FRET-peptide) having a P—N (proline-asparagine) scissile bond (P₁—P₁′) and having a G (glycine) in the P₂ position upstream of the P₁ proline. The FRET-peptide comprises a quenching group, e.g., DABCYL, on one side of the P₁P₁′ bond and a fluorophore group, e.g., EDANS, on the other side of the scissile bond. Preferably the FRET peptide has up to 13 amino acid residues upstream of the P₁ proline and up to 13 residues downstream of the P₁′ asparagine residue (i.e., the entire peptide including up to 28 amino acids). The amino acid which bears the quenching group (e.g., lysine) may be one of the up to 13 amino acids upstream or downstream of the P₁—P₁′ group and the amino acid which bears the fluorophore group (e.g., glutamic acid) may be one of the up to 13 amino acids upstream or downstream of the P₁—P₁′ group. Preferably the quenching and fluorophore groups are at the distal ends of the FRET peptide, and preferably at least one end of the peptide is terminated with an arginine residue (or other positively-charged amino acid), or alternatively one end may have a positively-charged amino acid and the other end may have a negatively-charged amino acid.

The terms “heterocycle” or “heterocyclic” refer to ring structures, preferably 4, 5, 6, or 7-membered ring structures, and more preferably 5- to 6-membered rings, whose ring structures include one to four heteroatoms such as nitrogen, sulfur, or oxygen. Heterocyclic groups include, but are not limited to, thiophene, furan, pyran, pyrrole, imidazole, pyrazole, isothiazole, isoxazole, pyridine, pyrazine, pyrimidine, and pyridazine.

The heterocyclic ring can be substituted at one or more positions with such substituents as, for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, CF₃, CN, or the like.

The term “carbocycle” or “carbocyclic”, as used herein, refers to an aromatic or non-aromatic ring in which each atom of the ring is carbon, preferably to 4, 5, 6 or 7 carbon rings, and more preferably to 5 and 6 carbon rings.

The proline analogs and derivatives of the peptidomimetic compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

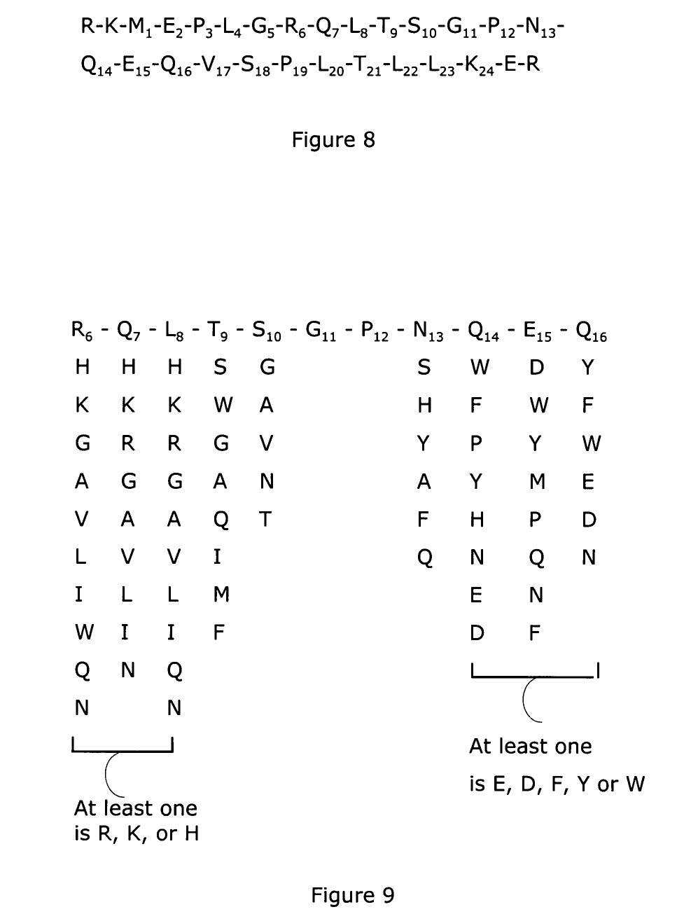

FIG. 8 shows an example of a FRET peptide (SEQ ID NO:5) having the native amino acid sequence surrounding the P₁—P₁′ bond of the Arg6 form of Met-α₂-AP. In this case the overall preferred maximal sequence of a FRET-peptide to be used for mimicking the substrate features of the Met-α₂-AP N-terminal peptide is shown. The rationale for including up to 13 residues on either side of the cleavage bond is to include like numbers of amino acid residues on either side of the P₁—P₁′ bond, since this level of symmetry minimizes potential steric problems as the enzyme and the substrate bind to cleave the P₁₂—N₁₃ (i.e., P₁—P₁′) bond. In this embodiment, optional arginine groups at each terminus enhance solubility of peptide derivatives when the —P—N— bond is cleaved. G₁₁ and P₁₂ (i.e., P₂—P₁) are preferred amino acids which cannot be substituted by other naturally occurring amino acids if the peptide is intended to be a cleavable substrate. The proline may be substituted with other proline-derivatives or analogs or proline-like amino acids in peptides intended for use as inhibitors. P₁₂—N₁₃— is the scissile bond. P₁₂, as noted, may be substituted to form an inhibitor. The fluorescent and quenching groups of the FRET Peptide are each coupled to amino acids, such as E and K near the termini in this embodiment. M₁ is the N-terminal methionine residue of the native sequence of precursor α₂AP.

FIG. 9 shows an alternate FRET peptide sequence (SEQ ID NO:6) having potential permutations of acceptable substitute amino acids at specific positions of a peptide comprising amino acids 8-18 of the peptide of FIG. 8.

In a preferred embodiment of the FRET peptide, the quenching group and the fluorophore group is each coupled to an amino acid residue which are on either side of the scissile bond and which are preferably within 1.0 to 5.0 nm of the scissile bond. In another preferred embodiment, each quenching and fluorophore groups is attached to any of the residues upstream of the P₂ glycine residue, or downstream of the P₁′ (asparagine or an appropriately substituted amino acid) residue, wherein the quenching group is on one side of the P₂ or P₁′ residue, and the fluorophore group is on the side of the P₂ or P₁′ residue, opposite the quenching group.

Experiments were performed using peptide substrates to investigate how structural variations (substitutions of amino acids) at and near the cleavage site of antiplasmin affect cleavage of antiplasmin by APCE.

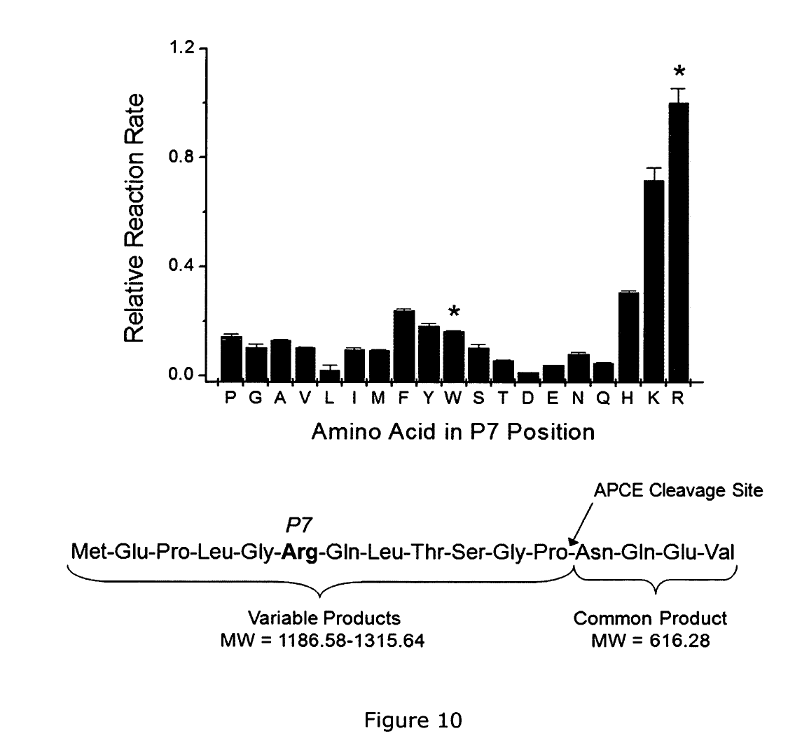

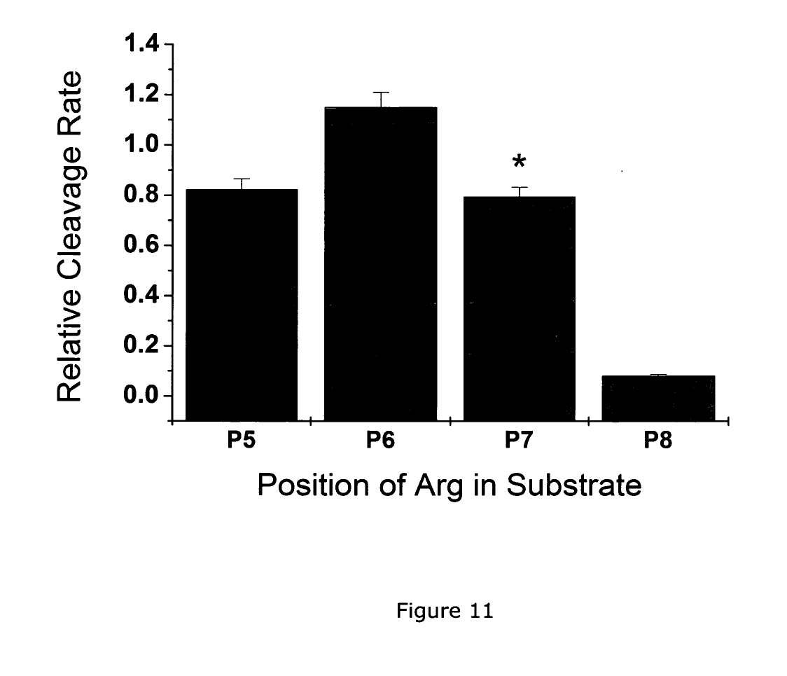

The amino acid sequence of Met-α₂AP surrounding the P₁—P₁′ bond cleaved by antiplasmin-cleaving enzyme (i.e., Pro-Asn) was used as a guide to produce identical synthetic peptides of varying length to determine residues important for enhancing cleavage of the peptide, assuming that cleavage rates are proportional to binding. Proline (or a carbocyclic or heterocyclic proline analog or substitute) at the P₁ position is a required residue for binding to APCE, as is glycine at the P₂ position upstream and adjacent the proline or proline substitute or analog residue. Working through a set of substitutions at various residues upstream from the P₁ proline, we found that a positively charged amino acid (e.g., arginine, lysine or histidine) at P₇ (i.e., the sixth residue upstream of the proline) or at positions P₅ or P₆ were particularly favorable for maximizing binding to APCE and thus the cleavage rate (FIGS. 10 and 11). Labeling shown herein in reference to P₅, P₆, P₇ refers to the residues as increasingly numbered from the Pro residue in the scissile bond in the N-terminal direction. Pro for example is P₁, with the subscript number increasing in the upstream direction.

FIG. 10 shows that an upstream arginine, or any positively charged amino acid, is critical for maximizing cleavage rate of the P₁—P₁′ bond by APCE in a peptide having SEQ ID NO:7. FIG. 11 indicates that by substituting arg in P₄, P₅, P₆ and P₈, we have determined that P₅ and P₆ are at least as effective as the arg in the native P₇ position, with P₆ being best. P₄ and P₈ were significantly inferior and showed no enhancement of the scissile bond cleavage. In addition to the presence of a positive charge in P₅, P₆, or P₇ having an accelerating effect on the scissile bond cleavage, there is also a spacing requirement for the positive charge effect to be maximal.

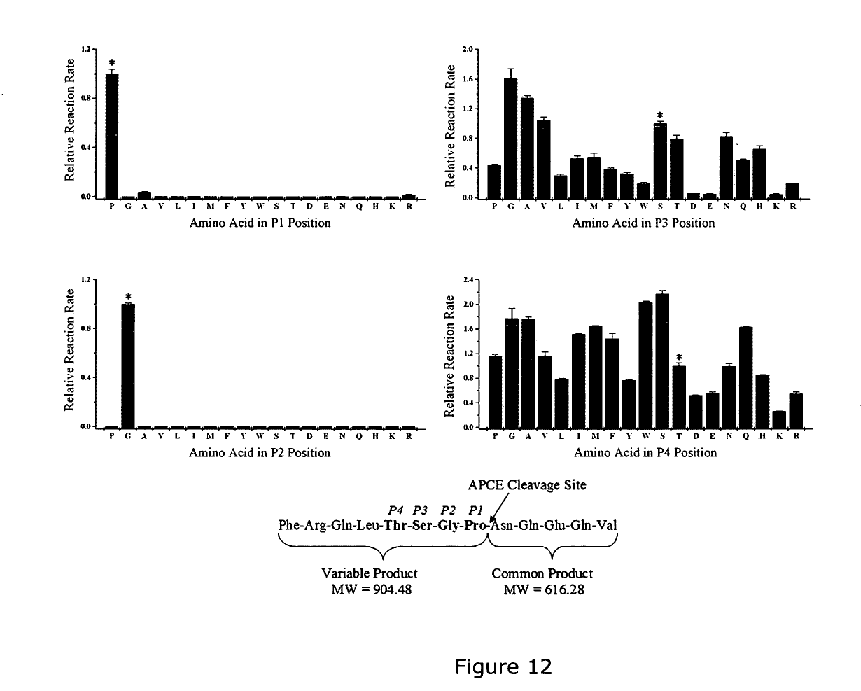

At position P₇ Arg was the optimal amino acid with a relative cleavage rate of about 5-10-fold faster than other amino acids except lys, which was about 70% of the arg rate (FIG. 10). The arginine enhancement effect at position P₇ (the sixth residue upstream from the P₁ proline) was also observed (FIG. 11) when arginine was in the P₆ or P₅ position (i.e., 4 or 5 residues upstream of the P₁—P₁′ bond), but cleavage rates diminished when arg was in positions P₈, P₄, P₃ or P₂ of the P₁—P₁′ bond (FIGS. 11 and 12). FIG. 12, for example, shows the effect of substituting each of 19 amino acids at each of amino acid sequence positions P₁—P₄ in a peptide (SEQ ID NO:8) and the effect on the reaction rate of cleavage of the P₁—P₁′ bond of the peptide by APCE. Positively charged residues, lys and his, can be substituted for arg in the P₇, P₆ and P₅ positions (see FIG. 10 for P₇), indicating the effect on cleavage rate of P₁—P₁′ is substantially positive-charge dependent (although pro, phe, tyr, trp, and some other amino acids have partial levels of activity (FIG. 10)).

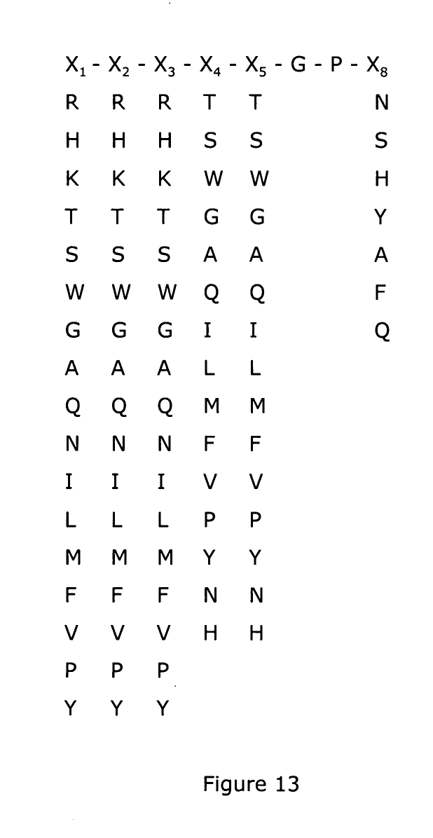

In an alternative embodiment, the FRET peptide of the present invention comprises a peptide sequence as shown in FIG. 13 (SEQ ID NO:9) wherein positions X₁-X₅ and X₈ may comprise an amino acid selected from the group of amino acids indicated below each of X₁-X₅ and X₈, with the proviso that at least one of X₁-X₃ is selected from R, H and K (i.e., is a positively charged amino acid). Further, at least one of X₁, X₂, and X₃ may be absent. As noted above, the FRET peptide comprising SEQ ID NO:9 (or any peptide or peptidomimetic) preferably comprises up to and including 28 amino acids.

The FRET peptide comprising SEQ ID NO:9 preferably comprises a quenching group and a fluorophore on opposite sides of the gly-pro bond. SEQ ID NO:9 may further comprise a 1-10mer peptide upstream of the N-terminal amino acid in the N-terminal direction and which may comprise any of the 20 natural amino acids, and may further comprise a 1-10mer peptide downstream of the C-terminal amino acid in the C-terminal direction and which may comprise any of the natural amino acids.

As noted above, the fluorescent resonance energy transfer peptide substrate may comprise the quenching group upstream of the P₁—P₁′ bond and the fluorophore downstream of the P₁—P₁′ bond, or the fluorescent resonance energy transfer peptide may comprise the quenching group downstream and the fluorophore upstream of the P₁—P₁′ bond. The P₁ is preferably proline or an effective proline analog or substitute, as described herein P₁′ is preferably asparagine, and a P₂ group upstream of P₁ is preferably glycine.

### Screening for APCE Inhibitors

In a particular embodiment, the invention comprises a method of screening for inhibitors of antiplasmin cleaving enzyme, comprising: providing a fluorescent resonance energy transfer peptide as contemplated herein, comprising a P₁—P₁′ bond and comprising a fluorophore (e.g., EDANS) and a quenching group (e.g., DABCYL) separated by the P₁—P₁′ bond such, that the peptide can be cleaved by α₂-antiplasmin cleaving enzyme, providing a quantity of α₂-antiplasmin cleaving enzyme, exposing the α₂-antiplasmin cleaving enzyme to an α₂-antiplasmin cleaving enzyme inhibitor candidate to form a test mixture, combining the test mixture with the fluorescent resonance energy transfer peptide, and measuring the fluorescence emission from the test mixture to identify when the activity of α₂-antiplasmin cleaving enzyme is inhibited by the α₂-antiplasmin cleaving enzyme inhibitor candidate.

### APCE Inhibitors

As described herein, various sequence variations of the FRET peptide and the peptide sequence surrounding the α₂AP scissile bond have been used herein for inhibitor development. In particular, substitutions of the proline of the scissile bond with proline analogs have led to development of specific APCE inhibitors as described herein. The present invention thus contemplates APCE-inhibitory peptidomimetics having up to 28 amino acids or more, and comprising proline analogs and derivatives for use as a P₁ proline substitute, including, but not limited to, the proline analogs and derivatives shown in Table I. The peptidomimetics preferably comprise 5, 6, 7, 8, 9, 10, 11, 12, 13, 14, 15, 16, 17, 18, 19, 20, 21, 22, 23, 24, 25, 26, 27, or 28 amino acids, not including spacer compounds for separating certain amino acids of the compound, or compounds which may be bound or complexed thereto for extending the serum life of the peptide, such as PEG or other polymeric materials or carriers.

An APCE inhibitor preferably possessing a positively charged residue at a distance corresponding to the length of four to seven residues upstream of the P₁—P₁′ scissile bond (3.5-24 Å, i.e., 0.35 nm-2.4 nm) can thus be used as a rapid, tight binding effective inhibitor of APCE. In addition, such inhibitors can be useful for inhibition of the closely related enzyme, fibroblast activation protein (FAP) and thus for the treatment of disorders relating to FAP, for example as described in U.S. Pat. No. 6,949,514.

The spacing of the positive charge in any of the P₅, P₆ or P₇ positions from the P₁—P₁′ scissile bond is a relevant determinant and therefore any amino acids or other spacers constructed of inert or neutral substances which fill this space to achieve the approximate length (i.e., 0.35 nm-2.4 nm) will be effective in constructing an APCE/FAP inhibitor. In a particularly favorable embodiment of the FRET peptide or APCE inhibitor, the arginine (or other positively-charged amino acid) is within from 6-21 Å (0.6 nm-2.1 nm) of the proline (or P₁ residue substitute) in the scissile bond.

The inhibitor in a preferred version comprises a sequence having 2-6 amino acids in the N-terminal direction from the P₁—P₁′ bond, including a positively-charged amino acid (e.g., arginine, lysine, or histidine), and preferably but not necessarily, includes a pro-asn linkage at P₁—P₁′ and preferably a glycine at P₂ and preferably a negatively-charged or aromatic amino acid (e.g., asp, glu, trp, tyr, and phe) at a position downstream (C-terminal direction) from the P₁—P₁′ bond.

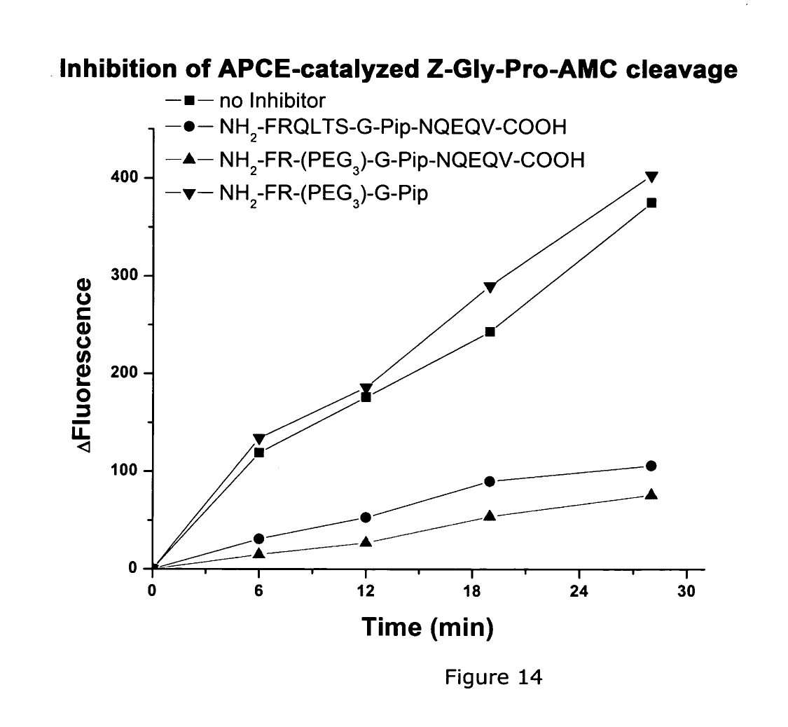

In a preferred embodiment of an inhibitor or FRET peptide of the present invention, the compound may comprise a spacer (linker or filler) group between the scissile bond and the positively-charged amino acid on the N-terminal side, wherein the positively-charged amino acid e.g., arg, his, or lys, is in a position equivalent to P₇, P₆ or P₅ (e.g., see FIG. 14) The spacer (i.e., linker or filler) between the scissile bond group and the positively charged amino acid in the P₅, P₆ or P₇, position may for example comprise a plurality of neutral, non-charged amino acids (e.g., glycine, alanine, leucine, isoleucine, valine, proline, methionine, tryptophan, tyrosine, threonine, serine, and most preferably serine, glycine, alanine, β-alanine, γ-amino butyric acid, epsilon amino caproic acid; or amino-PEG derivatives such as 8-, 11-, or 14-amino-3,6,9,12-tetraoxatetradecanoic acid and PEG oligomers (e.g., n=2-6)). These spacers may be homogenous (e.g., all glycine, alanine, etc., or other single amino acid) or heterogeneous (e.g., more than one type of amino acid or a hybrid amino acid/PEG molecule), and is preferably 3.5-21 Å (0.35 nm-2.1 nm) (or 1 to 7 amino acids) in length. The spacer may be comprised of neutral monomers such as ethylene glycol for example, or other similar monomer units (e.g., propylene glycol), which have a length of 3.5-21 Å (0.35 nm-2.1 nm) such that the spacer places the arginine (or other positively charged amino acid) within about 5-25 Å (0.5 nm-2.5 nm) of the proline or proline substitute or analog of the scissile bond.

Therefore the presence of a positively-charged amino acid within the distance as defined herein is an aspect of the invention that contributes significantly to the specificity of binding to the APCE sequence. Moreover, when amino acids within this space are substituted with polyethylene glycol of lengths of 3.5-21 Å (0.35 nm-2.1 nm), the cleavage rate of the P12—N13 bond remains maximal for the following peptide, X—R₆—(PEG₃)-G₁₁-P₁₂—N₁₃-Q₁₄-E₁₅ (SEQ ID NO:10) where (PEG₃) represents polyethylene glycol containing three ethylene glycol units and X is any amino acid. Given the specificity of the positive charge effect that must be within a certain distance from P₁₂ (P₁), this pegylated peptide is the basis for inhibitor development through selective modifications or substitutions of proline, P₁₂, for inhibiting the proteinase activity of APCE. FIG. 14 shows the APCE inhibitory effect of several peptides (SEQ ID NO:11-13) with the indicated sequences at 20 μM of each peptide when Pro12 is substituted with pipecolic acid, a proline analog.

In a preferred embodiment the invention comprises a compound having the formula:
Xaa₁-Sp₁-Gly-Cyc-Xaa₂-SP₂-Xaa₃ (Formula I).
In this embodiment Xaa₁ is a positively-charged amino acid, including but not limited to, arginine, histidine, or lysine. Sp₁ is a spacer molecule comprising 1 to 6 amino acids, preferably neutral amino acids including serine, glycine, alanine, or β-alanine, or gamma amino butyric acid, epsilon amino caproic acid, 8-amino-3,6,9,12-tetraoxatetradecanoic acid, 11-amino-3,6,9,12-tetraoxatetradecanoic acid, 14-amino-3,6,9,12-tetraoxatetradecanoic acid, or PEGₙ, or PPGₙ, where n=1-6 ethylene glycol or propylene glycol units, and wherein Sp₁ has a length of 3 Å to 21 Å (0.3 nm-2.1 nm). Gly is glycine. Cyc is a carbocyclic or heterocyclic ring. The carbocyclic ring may comprise 4, 5, 6, or 7 carbon atoms, and the heterocyclic ring may comprise 4, 5, 6, or 7 atoms, for example wherein at least one atom is a heteroatom such as nitrogen. Xaa₂ is preferably glutamine, asparagine, serine, histidine, tyrosine, alanine, or phenylalanine, or any natural amino acid which can serve as the P₁′ atom in the P₁—P₁′ scissle bond of the compound of the present invention. SP₂ is a spacer molecule and may be absent, or may comprise 1 to 6 amino acids, preferably neutral amino acids including serine, glycine, alanine, β-alanine, aspartic acid, glutamic acid, or gamma amino butyric acid, epsilon amino caproic acid, 8-amino-3,6,9,12-tetraoxatetradecanoic acid, 11-amino-3,6,9,12-tetraoxatetradecanoic acid, 14-amino-3,6,9,12-tetraoxatetradecanoic acid, or PEGₙ or PPGₙ, where n=1-6 ethylene glycol or propylene glycol units, and wherein Sp₁ has a length of 3 Å to 21 Å (0.3 nm-2.1 nm). Xaa₃ is glutamic acid, aspartic acid, tryptophan, tyrosine, or phenylalanine, or any negatively-charged amino acid, or aromatic amino acid. The compound may further comprise an N-terminal oligopeptide having 1 to 10 amino acids extending in an N-terminal direction from Xaa₁, and a C-terminal oligopeptide having 1-10 amino acids extending in a C-terminal direction from Xaa₃, wherein the N-terminal oligopeptide and C-terminal oligopeptide may comprise one or more of the 20 naturally-occurring amino acids. The compound is preferably disposed in a pharmaceutically-acceptable carrier as described elsewhere herein.

In one embodiment, the invention is a method of altering a plasma α₂-antiplasmin ratio in a subject having a pretreatment level of plasma Met-α₂-antiplasmin and a pretreatment level of plasma Asn-α₂-antiplasmin. In particular, the method comprises treating the subject with an inhibitor of antiplasmin cleaving enzyme wherein the inhibitor specifically inhibits cleavage of the Pro-Asn cleavage site of Met-α₂-antiplasmin by antiplasmin cleaving enzyme, wherein after treatment the subject has a posttreatment level of plasma Met-α₂-antiplasmin which is at least 50% greater than the pretreatment level of plasma Met-α₂-antiplasmin, and has a posttreatment level of plasma Asn-oα₂-antiplasmin which is at least 5% less than the pretreatment level of Asn-α₂-antiplasmin, thereby altering the plasma α₂-antiplasmin ratio in the subject. In the method, the alteration of the α₂-antiplasmin ratio in the subject preferably enhances fibrinolysis in the subject. In particular, the method is a treatment for inhibiting or treating atherosclerosis, arterial thromboses, venous thromboses, stroke, or pulmonary embolism.

In one embodiment of the method, the posttreatment level of plasma Met-α₂-antiplasmin is at least 10% greater than the pretreatment level of plasma Met-α₂-antiplasmin and the posttreatment level of plasma Asn-α₂-antiplasmin is at least 10% less than the pretreatment level of plasma Asn-α₂-antiplasmin. In another embodiment of the posttreatment level of plasma Met-α₂-antiplasmin is at least 15% greater than the pretreatment level of plasma Met-α₂-antiplasmin and the posttreatment level of plasma Asn-α₂-antiplasmin is at least 15% less than the pretreatment level of plasma Asn-α₂-antiplasmin. In another embodiment of the posttreatment level of plasma Met-α₂-antiplasmin is at least 20% greater than the pretreatment level of plasma Met-α₂-antiplasmin and the posttreatment level of plasma Asn-α₂-antiplasmin is at least 20% less than the pretreatment level of plasma Asn-α₂-antiplasmin. In another embodiment of the method, posttreatment level of plasma Met-α₂-antiplasmin is at least 25% greater (and may be, for example, any percentage greater) than the pretreatment level of plasma Met-α₂-antiplasmin and the posttreatment level of plasma Asn-α₂-antiplasmin is at least 25% less (and may be, for example, any percentage less) than the pretreatment level of plasma Asn-α₂-antiplasmin.

The invention preferably, in one embodiment, is a method of treating or inhibiting atherosclerosis, arterial thromboses, venous thromboses, stroke, or pulmonary embolism in a subject having a pretreatment level of plasma Met-α₂-antiplasmin and a pretreatment level of plasma Asn-α₂-antiplasmin by altering the α₂-antiplasmin level in the subject. The method comprises treating the subject with an inhibitor of antiplasmin cleaving enzyme wherein the inhibitor specifically inhibits cleavage of the Pro-Asn cleavage site of Met-α₂-antiplasmin by antiplasmin cleaving enzyme, wherein after treatment the subject has a posttreatment level of plasma Met-α₂-antiplasmin which is at least 5%, 10%, 15%, 20%, or 25% greater (or any percentage greater) than the pretreatment level of plasma Met-α₂-antiplasmin, and has a posttreatment level of plasma Asn-α₂-antiplasmin which is at least 5%, 10%, 15%, 20%, or 25% less (or any percentage less) respectively, than the pretreatment level of Asn-α₂-antiplasmin, thereby altering the plasma α₂-antiplasmin ratio in the subject. The alteration of α₂-antiplasmin ratio in the subject preferably occurs by enhancement of fibrinolysis in the subject.

The present invention further contemplates therapeutic compounds for treating cancers and disorders involving FAP such as those described in U.S. Pat. No. 6,949,514, which is expressly incorporated herein by reference. The therapeutic compounds comprise the APCE inhibitors of the present invention as described herein which are conjugated to carrier compounds which are able to pass through the cell membrane, including, but not limited to, protein transduction domains (PTDs). PTDs are positively charged peptides or peptide-like molecules that permeate cell membrane lipid bilayers. Typically PTDs contain several arginine residues and can be used to deliver other agents, such as peptides, proteins, oligonucleotides or small molecules through a cell membrane and into the cytosol. One PTD is a highly efficient molecular transporter formed by synthesizing an oligomer of arginines alternating with FACAs. Examples of PTDs which may be used in the present invention are shown in U.S. Pat. Nos. 7,166,692; 7,217,539; 7,053,200; 6,835,810; 6,645,501; and Published U.S. Patent Applications 2002/0009491; 2003/0032593; 2003/0162719; 2006/0159719; 2006/0293234; and 2007/0105775, each of which is expressly incorporated herein in its entirety by reference.

Utility

Utilities of the present invention include, but are not limited to:
(1) prevention or reduction of atherosclerotic plaque development and progression, especially in patients at high risk;
(2) prevention or reduction of the development of arterial or venous blood clot formation (atherothrombotic and venous thrombi disorders), especially in conditions recognized as high risk for such clots, i.e. heart attack or stroke;
(3) enhanced maintenance of vessel patency by continuous administration of the inhibitor of APCE, possibly in association with simultaneous administration of low doses of plasminogen activator drugs;
(4) prevention or reduction of fibrin formation where it may cause persistent acute or chronic symptoms in association with inflammatory conditions such as all forms of arthritis, organ fibrosis, undesirable scarring, and cancer and its metastases;
(5) reduction of the risk of bleeding as a hyperfibrinolytic state is induced, given that α₂AP_{act}, inhibits plasmin in solution state as well as α₂AP_{act}, when not crosslinked into fibrin;
(6) aiding in the prevention and therapy of fibrin deposits interfering with organ function as might be seen in atherothrombotic disease, such as coronary artery thrombosis, stroke, pulmonary embolism, all other forms of arterial and venous thromboses, inflammatory conditions, and cancer and its metastases;
(7) determining whether a subject has a Trp6 or Arg6 polymorphism in α₂-antiplasmin; and
(8) screening for inhibitors of antiplasmin cleaving enzyme.

Various embodiments of the invention therefore include, but are not limited to:
(1) a therapeutic method of promoting fibrin digestion in vivo, comprising administering to a subject an effective quantity of an inhibitor of antiplasmin cleaving enzyme;
(2) fluorescent resonance energy transfer (FRET) peptides for acting as substrates of APCE;
(3) an inhibitor of antiplasmin cleaving enzyme, wherein the inhibitor may comprise, or is linked to a polymeric spacer, linker, or carrier;
(4) an inhibitor of antiplasmin cleaving enzyme which is effective in binding to or blocking the α₂-antiplasmin binding site, or α₂-antiplasmin pro-asn cleaving site of antiplasmin cleaving enzyme;
(5) a method of enhancing fibrin digestion in vivo, comprising providing to a subject in need of clot digestion or clot prevention, simultaneously or in sequence, a quantity of plasminogen activator and an inhibitor of antiplasmin cleaving enzyme, wherein the quantity of plasminogen activator is less than the amount provided in standard therapeutic protocol absent the inhibitor of antiplasmin cleaving enzyme; and
(6) a DNA encoding the FRET peptide or inhibitor peptide contemplated herein, and a plasmid vector or host containing the DNA.

Other embodiments of the present invention provide pharmaceutically acceptable compositions which comprise a therapeutically-effective amount of one or more of the compounds described herein, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, aqueous or non-aqueous solutions or suspensions, tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set forth above, certain embodiments of the present compounds contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

As noted above, formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides.

Methods of preparing these formulations or compositions may include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, or paste.

In solid dosage forms of the invention for oral administration (capsules, tablets, pills, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subeuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, aqueous or non-aqueous solutions or suspensions, tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or oral cavity; or (4) intravaginally or intravectally, for example, as a pessary, cream or foam; (5) sublingually; (6) ocularly; (7) transdermally; or (8) nasally.

The compounds according to the invention may be formulated for administration in any convenient way for use in human or veterinary medicine, by analogy with other pharmaceuticals.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

As used herein, the term “subject” or “patient” preferably refers to a warm blooded animal such as a mammal which is afflicted with a particular inflammatory disease state. It is understood that guinea pigs, dogs, cats, rats, mice, horses, cattle, sheep, and humans are examples of animals within the scope of the meaning of the term.

A therapeutically effective amount of the compound used in the treatment described herein can be readily determined by the attending diagnostician, as one skilled in the art, by the use of conventional techniques and by observing results obtained under analogous circumstances. In determining the therapeutically effective dose, a number of factors are considered by the attending diagnostician, including, but not limited to: the species of mammal; its size, age, and general health; the specific disease or condition involved; the degree of or involvement or the severity of the disease or condition; the response of the individual subject; the particular compound administered; the mode of administration; the bioavailability characteristic of the preparation administered; the dose regimen selected; the use of concomitant medication; and other relevant circumstances.

A therapeutically effective amount of the compositions of the present invention will generally contain sufficient active ingredient (i.e., the APCE or inhibitor thereof) to deliver from about 0.1 μg/kg to about 100 mg/kg (weight of active ingredient/body weight of patient). Preferably, the composition will deliver at least 0.5 μg/kg to 50 mg/kg, and more preferably at least 1 μg/kg to 10 mg/kg.

Practice of the method of the present invention comprises administering to a subject a therapeutically effective amount of the active ingredient, in any suitable systemic or local formulation, in an amount effective to deliver the dosages listed above. The dosage can be administered on a one-time basis, or (for example) from one to five times per day or once or twice per week, or continuously via a venous drip, or other means, depending on the desired therapeutic effect.

As noted, preferred amounts and modes of administration are able to be determined by one skilled in the art. One skilled in the art of preparing formulations can readily select the proper form and mode of administration depending upon the particular characteristics of the compound selected, the disease state to be treated, the stage of the disease, and other relevant circumstances using formulation technology known in the art, described, for example, in Remington's Pharmaceutical Sciences, latest edition, Mack Publishing Co.

Pharmaceutical compositions can be manufactured utilizing techniques known in the art. Typically the therapeutically effective amount of the compound will be admixed with a pharmaceutically acceptable carrier.

The half-life of the molecules described herein can be extended by their being conjugated to other molecules such as polymers using methods known in the art to form drug-polymer conjugates. For example, the molecules can be bound to molecules of inert polymers known in the art, such as a molecule of polyethylene glycol (PEG) in a method known as “pegylation”. Pegylation can therefore extend the in vivo lifetime and thus therapeutic effectiveness of the molecule.

PEG molecules can be modified by functional groups, for example as shown in Harris et al., “Pegylation, A Novel Process for Modifying Phararmacokinetics”, Clin Pharmacokinet, 2001: 40(7); 539-551, and the amino terminal end of the molecule, or cysteine residue if present, or other linking amino acid therein can be linked thereto, wherein the PEG molecule can carry one or a plurality of one or more types of molecules.

By “polyethylene glycol” or “PEG” is meant a polyalkylene glycol compound or a derivative thereof, with or without coupling agents or derivatization with coupling or activating moeities (e.g., with thiol, triflate, tresylate, azirdine, oxirane, or preferably with a maleimide moiety). Compounds such as maleimido monomethoxy PEG are exemplary or activated PEG compounds of the invention. Other polyalkylene glycol compounds, such as polypropylene glycol, may be used in the present invention. Other appropriate polymer conjugates include, but are not limited to, non-polypeptide polymers, charged or neutral polymers of the following types: dextran, colominic acids or other carbohydrate based polymers, biotin deriviatives and dendrimers, for example. The term PEG is also meant to include other polymers of the class polyalkylene oxides.

The PEG can be linked to any N-terminal amino acid of the molecule described herein and/or can be linked to an amino acid residue downstream of the N-terminal amino acid, such as lysine, histidine, tryptophan, aspartic acid, glutamic acid, and cysteine, for example or other such amino acids known to those of skill in the art.

The PEG carrier moiety attached to the peptide may range in molecular weight from about 200 to 20,000 MW. Preferably the PEG moiety will be from about 1,000 to 8,000 MW, more preferably from about 3,250 to 5,000 MW, most preferably about 5,000 MW. The actual number of PEG molecules covalently bound per molecule of the invention may vary widely depending upon the desired stability (i.e. serum half-life). Molecules contemplated herein can be linked to PEG molecules using techniques shown, for example (but not limited to), in U.S. Pat. Nos. 4,179,337; 5,382,657; 5,972,885; 6,177,087; 6,165,509; 5,766,897; and 6,217,869; the specifications and drawings each of which are hereby expressly incorporated herein by reference.

Alternatively, it is possible to entrap the molecules in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are disclosed in the latest edition of Remington's Pharmaceutical Sciences.

U.S. Pat. No. 4,789,734 describe methods for encapsulating biochemicals in liposomes and is hereby expressly incorporated by reference herein. Essentially, the material is dissolved in an aqueous solution, the appropriate phospholipids and lipids added, along with surfactants if required, and the material dialyzed or sonicated, as necessary. A review of known methods is by G. Gregoriadis, Chapter 14. “Liposomes”, Drug Carriers in Biology and Medicine, pp. 287-341 (Academic Press, 1979). Microspheres formed of polymers or proteins are well known to those skilled in the art, and can be tailored for passage through the gastrointestinal tract directly into the blood stream. Alternatively, the agents can be incorporated and the microspheres, or composite of microspheres, implanted for slow release over a period of time, ranging from days to months. See, for example, U.S. Pat. Nos. 4,906,474; 4,925,673; and 3,625,214 which are incorporated by reference herein.

When the composition is to be used as an injectable material, it can be formulated into a conventional injectable carrier. Suitable carriers include biocompatible and pharmaceutically acceptable phosphate buffered saline solutions, which are preferably isotonic.

For reconstitution of a lyophilized product in accordance with this invention, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field.

The pharmaceutical composition may also be in the form of an aqueous solution containing many of the same substances as described above for the reconstitution of a lyophilized product.

The compounds can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

As mentioned above, the compounds of the invention may be incorporated into pharmaceutical preparations which may be used for therapeutic purposes. However, the term “pharmaceutical preparation” is intended in a broader sense herein to include preparations containing a glycosulfopeptide composition in accordance with this invention, used not only for therapeutic purposes but also for reagent or diagnostic purposes as known in the art, or for tissue culture. The pharmaceutical preparation intended for therapeutic use should contain a “pharmaceutically acceptable” or “therapeutically effective amount” of a molecule as defined herein.

All of the assay methods listed herein are well within the ability of one of ordinary skill in the art given the teachings provided herein.

All references, patents and patent applications cited herein are hereby incorporated herein in their entireties by reference. In particular, U.S. Ser. Nos. 10/774,242, 60/445,774, 60/811,568, and 60/836,365 are hereby expressly incorporated herein by reference in their entireties.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, manufacture, compositions of matter, means, methods and steps described in the specification. As one of ordinary skill in the art will readily appreciate from the disclosure of the present invention, processes, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, manufacture, compositions of matter, means, methods, or steps.

### REFERENCES

Collen D. The plasminogen (fibrinolytic) system. Thromb. Haemost. 1999; 82:259-270.
Wiman B, Collen D. On the kinetics of the reaction between human antiplasmin and plasmin. Eur. J. Biochem. 1978; 84:573-578.
Mullertz S, Clemmensen I. The primary inhibitor of plasmin in human plasma. Biochem. J. 1976; 159:545-553.
Collen D. Identification and some properties of a new fast-reacting plasmin inhibitor in human plasma. Eur. J. Biochem. 1976; 69:209-216.
Bangert K, Johnsen A H, Christensen U, Thorsen S. Different N-terminal forms of alpha 2-plasmin inhibitor in human plasma. Biochem. J. 1993; 291 (Pt 2):623-625.
Koyama T, Koike Y, Toyota S et al. Different NH2-terminal form with 12 additional residues of alpha 2-plasmin inhibitor from human plasma and culture media of Hep G2 cells. Biochem. Biophys. Res. Commun. 1994; 200:417-422.
Lee K N, Jackson K W, Christiansen V J, Chung K H, McKee P A. A novel plasma proteinase potentiates alpha2-antiplasmin inhibition of fibrin digestion. Blood 2004; 103:3783-3788.
Lee K N, Jackson K W, Christiansen V J et al. Antiplasmin-cleaving enzyme is a soluble form of fibroblast activation protein. Blood 2006; 107:1397-1404.
Hirosawa S, Nakamura Y, Miura O, Sumi Y, Aoki N. Organization of the human alpha 2-plasmin inhibitor gene. Proc. Natl. Acad. Sci. U.S.A 1988; 85:6836-6840.
Holmes W E, Nelles L, Lijnen H R, Collen D. Primary structure of human alpha 2-antiplasmin, a serine protease inhibitor (serpin). J. Biol. Chem. 1987; 262:1659-1664.
Tone M, Kikuno R, Kume-Iwaki A, Hashimoto-Gotoh T. Structure of human alpha 2-plasmin inhibitor deduced from the cDNA sequence. J. Biochem. (Tokyo) 1987; 102:1033-1041.
Lind B, Thorsen S. A novel missense mutation in the human plasmin inhibitor (alpha2-antiplasmin) gene associated with a bleeding tendency. Br. J. Haematol. 1999; 107:317-322.
Wiman B. Affinity-chromatographic purification of human alpha 2-antiplasmin. Biochem. J. 1980; 191:229-232.
Beebe D P, Aronson D L. An automated fibrinolytic assay performed in microtiter plates. Thromb. Res. 1987; 47:123-128.
Jones A J, Meunier A M. A precise and rapid microtitre plate clot lysis assay: methodology, kinetic modeling and measurement of catalytic constants for plasminogen activation during fibrinolysis. Thromb. Haemost. 1990; 64:455-463.
Lee K N, Lee S C, Jackson K W et al. Effect of phenylglyoxal-modified alpha2-antiplasmin on urokinase-induced fibrinolysis. Thromb. Haemost. 1998; 80:637-644.
Tam J P. Synthetic peptide vaccine design: synthesis and properties of a high-density multiple antigenic peptide system. Proc. Natl. Acad. Sci. U.S.A 1988; 85:5409-5413.
von Rokitansky C. Abnormal conditions of the arteries. A Manual of Pathological Anatomy. London: Sydenham Society; 1852: 261-275.
Virchow R. Phlogose und Thrombose im Gefasssystem. In: Virchow R, ed. Gesammelte Abhandlungen zur Wissenschatflichen Medizin. Frankfurt: Meidinger Sohn & Co.; 1856: 458-636.
Smith E B. Fibrinogen, fibrin and fibrin degradation products in relation to atherosclerosis. Clin. Haematol. 1986; 15:355-370.
Jorgensen L. The role of platelets in the initial stages of atherosclerosis. J. Thromb. Haemost. 2006; 4:1443-1449.
Schwartz C J, Valente A J, Kelley J L, Sprague E A, Edwards E H. Thrombosis and the development of atherosclerosis: Rokitansky revisited. Semin. Thromb. Hemost. 1988; 14:189-195.
Fuster V, Badimon L, Badimon J J, Chesebro J H. The pathogenesis of coronary artery disease and the acute coronary syndromes (1). N. Engl. J. Med. 1992; 326:242-250.
Bini A, Kudryk B J. Fibrinogen in human atherosclerosis. Ann. N.Y. Acad. Sci. 1995; 748:461-471.
Bini A, Fenoglio J J, Jr., Mesa-Tejada R, Kudryk B, Kaplan K L. Identification and distribution of fibrinogen, fibrin, and fibrin(ogen) degradation products in atherosclerosis. Use of monoclonal antibodies. Arteriosclerosis 1989; 9:109-121.
Loscalzo J. The relation between atherosclerosis and thrombosis. Circulation 1992; 86:III95-III99.
Smith E B, Keen G A, Grant A, Stirk C. Fate of fibrinogen in human arterial intima. Arteriosclerosis 1990; 10:263-275.
Smith E B, Thompson W D. Fibrin as a factor in atherogenesis. Thromb. Res. 1994; 73:1-19.
White J G. Platelets and atherosclerosis. Eur. J. Clin. Invest 1994; 24 Suppl 1:25-29.
Xiao Q, Danton M J, Witte D P et al. Fibrinogen deficiency is compatible with the development of atherosclerosis in mice. J. Clin. Invest 1998; 101:1184-1194.
Marutsuka K, Hatakeyama K, Yamashita A, Asada Y. Role of thrombogenic factors in the development of atherosclerosis. J. Atheroscler. Thromb. 2005; 12:1-8.
Kathiresan S, Yang Q, Larson M G et al. Common genetic variation in five thrombosis genes and relations to plasma hemostatic protein level and cardiovascular disease risk. Arterioscler. Thromb. Vasc. Biol. 2006; 26:1405-1412.
Spurlock B O, Chandler A B. Adherent platelets and surface microthrombi of the human aorta and left coronary artery: a scanning electron microscopy feasibility study. Scanning Microsc. 1987; 1:1359-1365.
Meade T W, Ruddock V, Stirling Y, Chakrabarti R, Miller G J. Fibrinolytic activity, clotting factors, and long-term incidence of ischaemic heart disease in the Northwick Park Heart Study. Lancet 1993; 342:1076-1079.
Xiao Q, Danton M J, Witte D P et al. Plasminogen deficiency accelerates vessel wall disease in mice predisposed to atherosclerosis. Proc. Natl. Acad. Sci. U.S.A 1997; 94:10335-10340.
Kwaan H C. Physiologic and pharmacologic implications of fibrinolysis. Artery 1979; 5:285-290.
Smith E B. Haemostatic factors and atherogenesis. Atherosclerosis 1996; 124:137-143.
Juhan-Vague I, Collen D. On the role of coagulation and fibrinolysis in atherosclerosis. Ann. Epidemiol. 1992; 2:427-438.
Tanaka K, Sueishi K. The coagulation and fibrinolysis systems and atherosclerosis. Lab Invest 1993; 69:5-18.
Favier R, Aoki N, de M P. Congenital alpha(2)-plasmin inhibitor deficiencies: a review. Br. J. Haematol. 2001; 114:4-10.
Eda K, Ohtsuka S, Seo Y et al. Conservative treatment of hemolytic complication following coil embolization in two adult cases of patent ductus arteriosus. Jpn. Circ. J. 2001; 65:834-836.
Levi M, Roem D, Kamp A M et al. Assessment of the relative contribution of different protease inhibitors to the inhibition of plasmin in vivo. Thromb. Haemost. 1993; 69:141-146.
Harpel P C, Mosesson M W. Degradation of human fibrinogen by plasmas alpha2-macroglobulin-enzyme complexes. J. Clin. Invest 1973; 52:2175-2184.

Aoki N, Harpel P C. Inhibitors of the fibrinolytic enzyme system. Semin. Thromb. Hemost. 1984; 10:24-41.

TABLE I
Cyclic Amines and Proline Analogs

4-hydroxypyrrolidine-2-carboxylic acid (cis and trans)
3-phenylpyrrolidine-2-carboxylic acid (cis and trans)
3-hydroxypyrrolidine-2-carboxylic acid (cis and trans)
4-hydroxypyrrolidine-2-carboxylic acid (cis and trans)
2-ethylthiazolidine-4-carboxylic acid (cis and trans)
2-methylthiazolidine-4-carboxylic acid (cis and trans)
2-phenylthiazolidine-4-carboxylic acid (cis and trans)
5,5-dimethylthiazolidine-4-carboxylic acid
thiazolidine-2-carboxylic acid (cis and trans)
thiazolidine-4-carboxylic acid (cis and trans)
azetidine-2-carboxylic acid (cis and trans)
thiazolidine-2-carboxylic acid (cis and trans)
thiazolidine-4-carboxylic acid (cis and trans)
amino-L-proline methyl ester
cyano-L-proline methyl ester
4-cyano-L-proline
3,4-dehydro-L-proline
4-fluoro-L-proline
lysyl piperidide
N-(4-chlorobenzyl)4-0x0-4-(1-piperidinyl)-1,3-(s)-butane-diamine
bromocyclopentyl carboxylic acid
chlorocyclopentyl carboxylic acid
fluorocyclopentyl carboxylic acid
cis-3-methylproline
cis-3-ethylproline
cis-3-isopropylproline
cis-3-isopentanylproline
homoproline
benzyl-proline
(2-fluoro-benzyl)-proline
(3-fluoro-benzyl)-proline
(4-fluoro-benzyl)-proline
(2-chloro-benzyl)-proline
(3-chloro-benzyl)-proline
(4-chloro-benzyl)-proline
(2-bromo-benzyl)-proline
(3-bromo-benzyl)-proline
(4-bromo-benzyl)-proline
phenethyl-proline
(2-methyl-benzyl)-proline
(3-methyl-benzyl)-proline
(4-methyl-benzyl)-proline
(2-nitro-benzyl)-proline
(3-nitro-benzyl)-proline
(4-nitro-benzyl)-proline
(1-Naphthalenylmethyl)-proline
(2-Naphthalenylmethyl)-proline
(2,4-dichloro-benzyl)-proline
(3,4-dichloro-benzyl)-proline
(3,4-difluoro-benzyl)-proline
(2-trifluoromethyl-benzyl)-proline
(3-trifluoromethyl-benzyl)-proline
(4-trifluoromethyl-benzyl)-proline
(2-cyano-benzyl)-proline
(3-cyano-benzyl)-proline
(4-cyano-benzyl)-proline
(4-iodo-benzyl)-proline
(3-Phenyl-allyl)-proline
(3-Phenyl-allyl)-proline
(3-Phenyl-propyl)-proline
(4-tert-Butyl-benzyl)-proline
Benzhydryl-proline
(4-Biphenylmethyl)-proline
(4-Thiazolylmethyl)-proline
(3-Benzo[b]thiophenylmethyl)-proline
(2-Thiophenylmethyl)-proline
(5-Bromo-2-Thiophenylmethyl)-proline
(3-Thiophenylmethyl)-proline
(2-Furanylmethyl)-proline
(2-Pyridinylmethyl)-proline
(3-Pyridinylmethyl)-proline
(4-Pyridinylmethyl)-proline
Allyl-proline
Propynyl-proline
4-Phenyl-pyrrolidine-3-carboxylic acid
4-(2-fluoro-phenyl)-pyrrolidine-3-carboxylic acid
4-(3-fluoro-phenyl)-pyrrolidine-3-carboxylic acid
trans-4-(4-fluoro-phenyl)-pyrrolidine-3-carboxylic acid
trans-4-(2-chloro-phenyl)-pyrrolidine-3-carboxylic acid
trans-4-(3-chloro-phenyl)-pyrrolidine-3-carboxylic acid
4-(4-chloro-phenyl)-pyrrolidine-3-carboxylic acid
4-(2-bromo-phenyl)-pyrrolidine-3-carboxylic acid
4-(3-bromo-phenyl)-pyrrolidine-3-carboxylic acid
trans-4-(4-bromo-phenyl)-pyrrolidine-3-carboxylic acid
4-(2-methyl-phenyl)-pyrrolidine-3-carboxylic acid
4-(3-methyl-phenyl)-pyrrolidine-3-carboxylic acid
4-(4-methyl-phenyl)-pyrrolidine-3-carboxylic acid
4-(2-nitro-phenyl)-pyrrolidine-3-carboxylic acid
4-(3-nitro-phenyl)-pyrrolidine-3-carboxylic acid
4-(4-nitro-phenyl)-pyrrolidine-3-carboxylic acid
4-(1-naphthyl)-pyrrolidine-3-carboxylic acid
4-(2-naphthyl)-pyrrolidine-3-carboxylic acid
4-(2,5-dichloro-phenyl)-pyrrolidine-3-carboxylic acid
4-(2,3-dichloro-phenyl)-pyrrolidine-3-carboxylic acid
4-(2-trifluoromethyl-phenyl)-pyrrolidine-3-carboxylic acid
4-(3-trifluoromethyl-phenyl)-pyrrolidine-3-carboxylic acid
4-(4-trifluoromethyl-phenyl)-pyrrolidine-3-carboxylic acid
4-(2-cyano-phenyl)-pyrrolidine-3-carboxylic acid
4-(3-cyano-phenyl)-pyrrolidine-3-carboxylic acid
4-(4-cyano-phenyl)-pyrrolidine-3-carboxylic acid
4-(2-methoxy-phenyl)-pyrrolidine-3-carboxylic acid
4-(3-methoxy-phenyl)-pyrrolidine-3-carboxylic acid
4-(4-methoxy-phenyl)-pyrrolidine-3-carboxylic acid
4-(2-hydroxy-phenyl)-pyrrolidine-3-carboxylic acid
4-(3-hydroxy-phenyl)-pyrrolidine-3-carboxylic acid
4-(4-hydroxy-phenyl)-pyrrolidine-3-carboxylic acid
4-(2,3-dimethoxy-phenyl)-pyrrolidine-3-carboxylic acid
4-(3,4-dimethoxy-phenyl)-pyrrolidine-3-carboxylic acid
4-(3,5-dimethoxy-phenyl)-pyrrolidine-3-carboxylic acid
4-(2-pyridinyl)-pyrrolidine-3-carboxylic acid
4-(3-pyridinyl)-pyrrolidine-3-carboxylic acid
4-(6-methoxy-3-pyridinyl)-pyrrolidine-3-carboxylic acid
4-(4-pyridinyl)-pyrrolidine-3-carboxylic acid
4-(2-thienyl)-pyrrolidine-3-carboxylic acid
4-(3-thienyl)-pyrrolidine-3-carboxylic acid
4-(2-furanyl)-pyrrolidine-3-carboxylic acid
4-isopropyl-pyrrolidine-3-carboxylic acid
piperidine
pyrrolidine
azetidine
pipecolic acid
piperidide
3-carboxy-1,2,3,4-tetrahydro-isoquinoline
2-carboxy-2,3-dehydroindole
acetyl-gly-valboropro
cyclopentyls
N-substituted cyclopentyl derivatives
cyclohexyls
N-substituted cyclohexyl derivatives
val-boropro
glu-boropro
oxazolidine
and proline analogs and derivatives as defined in U.S. Pat.
No. 4,428,939; 6,890,904; 4,762,821 and Published
Applications 2003/0158114; 20050272703; and
2006/0287245.

What is claimed is:

1. A compound having the formula:
Xaa₁-Sp₁-Gly-Cyc-Xaa₂-Sp₂-Xaa₃
wherein:
Xaa₁ is arginine, histidine, or lysine;
Sp₁ is a spacer comprising at least one of 8-, 11-, or 14-amino-3,6,9,12-tetraoxatetradecanoic acid, PEGₙ or PPGₙ wherein n=1-6, gamma amino butyric acid, epsilon amino caproic acid, serine, glycine, alanine, or β-alanine, or a combination thereof, wherein Sp₁ has a length of 0.3 nm-2.1 nm;
Gly is glycine;
Cyc is a carbocyclic or heterocyclic compound;
Xaa₂ is glutamine, asparagine, serine, histidine, tyrosine, alanine, or phenylalanine;
SP₂ is a spacer comprising at least one of PEGₙ or PPGₙ where n=1-5, 8-, 11-, or 14-amino-3,6,9,12-tetraoxatetradecanoic acid, gamma amino butyric acid, epsilon amino caproic acid, serine, glycine, alanine, β-alanine, aspartic acid, glutamic acid, or a combination thereof, or is absent; and
Xaa₃ is glutamic acid, aspartic acid, tryptophan, tyrosine, or phenylalanine.

2. The compound of claim 1 wherein Cyc is a 4, 5, 6, or 7-member carbon carbocycle.

3. The compound of claim 1 wherein Cyc is a 4, 5, 6, or 7-member carbon heterocycle.

4. The compound of claim 3 wherein the carbon heterocycle comprises a nitrogen heteroatom.

5. The compound of claim 1 wherein Sp₁ is PEGₙ and wherein n=2-5.

6. The compound of claim 1 wherein Sp₁ has a length of 0.6 nm to 17.5 nm.

7. The compound of claim 1 comprising a 1-8mer oligopeptide extending from Xaa₁ in the N-terminal direction.

8. The compound of claim 1 comprising a 2-10mer oligopeptide extending from Xaa₃ in the C-terminal direction.

9. The compound of claim 1 disposed within a pharmaceutically-acceptable carrier.

10. The compound of claim 1 linked via an amino acid residue to a polyethylene glycol carrier molecule.

11. A method of screening for inhibitors of antiplasmin cleaving enzyme, comprising:
providing a fluorescent resonance energy transfer peptide which is cleavable by α₂-antiplasmin cleaving enzyme, and which comprises a P₁—P₁′ bond, and comprises a fluorophore and a quenching group separated by the P₁—P₁′ bond, wherein the P₁ comprises a proline, the P₁′ comprises an asn, phe, gin, ser, tyr, his or ala, and having a P₂ residue comprising gly;
providing a quantity of α₂-antiplasmin cleaving enzyme;
exposing the α₂-antiplasmin cleaving enzyme to an α₂-antiplasmin cleaving enzyme inhibitor candidate to form a test mixture;
combining the test mixture with the fluorescent resonance energy transfer peptide; and
measuring the fluorescence emission from the test mixture for identifying when the α₂-antiplasmin cleaving enzyme inhibitor candidate inhibits the activity of α₂-antiplasmin cleaving enzyme.

12. The method of claim 11 wherein the fluorescent resonance energy transfer peptide comprises the quenching group upstream of the proline-asparagine bond and the fluorophore downstream of the P₁—P₁′ bond.

13. The method of claim 11 wherein the fluorescent resonance energy transfer peptide comprises the quenching group downstream and the fluorophore upstream of the P₁—P₁′ bond.

14. The method of claim 11 wherein the fluorescent resonance energy transfer peptide comprises SEQ ID NO:9.

15. An inhibitor of oα₂-antiplasmin cleaving enzyme identified by the screening method of claim 11.

16. An oligopeptide substrate of α₂-antiplasmin cleaving enzyme, comprising:
X₁-X₂-X₃-X₄-X₅-Gly-Pro-X₈ (SEQ ID NO:9)
wherein:
X₁ is selected from arg, his, lys, thr, ser, trp, gly, ala, gin, asn, ile, leu, met, phe, val, pro, and tyr;
X₂ is selected from arg, his, lys, thr, ser, trp, gly, ala, gin, asn, ile, leu, met, phe, val, pro, and tyr;
X₃ is selected from arg, his, lys, thr, ser, trp, gly, ala, gin, asn, ile, leu, met, phe, val, pro, and tyr;
X₄ is selected from thr, ser, trp, gly, ala, gin, ile, leu, met, phe, val, pro, tyr, asn, and his;
X₅ is selected from thr, ser, trp, gly, ala, gin, ile, leu, met, phe, val, pro, tyr, asn, and his;
X₈ is selected from asn, ser, his, tyr, ala, phe, gin;
and wherein at least one of X₁, X₂, and X₃ is selected from arg, his, and lys;
and wherein one or two of X₁, X₂, and X₃ may be absent and wherein the oligopeptide consists of up to 28 amino acids.

17. The oligopeptide of claims 16 further comprising a 1-8mer oligopeptide extending from X₁ in the N-terminal direction.

18. The oligopeptide of claim 16 further comprising a 2-10mer oligopeptide extending from X₈ in the C-terminal direction.

19. The oligopeptide of claim 16 further comprising a quenching group and a fluorophore on opposite sides of the Gly-Pro bond.


SEQUENCE LISTING (12 sequences)

SEQ ID NO: 1 - DNA, 22 bp, Homo sapiens
gacctcctatcctcatcccttt

SEQ ID NO: 2 - DNA, 21 bp, Homo sapiens
ctggttcggcccgctagttag

SEQ ID NO: 3 - protein, 12 aa, Homo sapiens
MEPLGRQLTSGP

SEQ ID NO: 4 - protein, 12 aa, Homo sapiens
MEPLGWQLTSGP

SEQ ID NO: 5 - protein, 28 aa, Homo sapiens
RKMEPLGRQLTSGPNQEQVSPLTLLKER

SEQ ID NO: 6 - protein, 11 aa, Homo sapiens
  MISC_FEATURE (1)..(1): Other Information  Xaa at position 1 is selected from arg, gln, his, asn, lys, gly, ala, val, ile, or leu, wherein at least oneof Xaa(1), Xaa(2), and Xaa(3) is one of arg, his, or lys.
  MISC_FEATURE (2)..(2): Other Information Xaa at position 2 is selected from gln, his, asn, lys, arg, gly, ala, val, ile, or leu, wherein at least oneof Xaa(1), Xaa(2), and Xaa(3) is one of arg, his, or lys.
  MISC_FEATURE (3)..(3): Other Information  Xaa at position 3 is selected from leu, his, asn, gln, lys, arg, gly, ala, val, ile, or leu, wherein at least one of Xaa(1), Xaa(2), and Xaa(3) is one of arg, his, or lys.
  MISC_FEATURE (3)..(3): Other Information  Xaa at position 3 is selected from leu, his, asn, gln, lys, arg, gly, ala, val, ile, or leu, wherein at least one of Xaa(1), Xaa(2), and Xaa(3) is one of arg, his, or lys.
  MISC_FEATURE (4)..(4): Other Information  Xaa at position 4 is selected from thr, ser, trp, gly, ala, gln, ile, met, or phe.
  MISC_FEATURE (5)..(5): Other Information  Xaa at position 5 is selected from ser, gly, ala, val, asn, or thr.
  MISC_FEATURE (8)..(8): Other Information  Xaa at position 8 is selected from asn, ser, his, tyr, ala, phe, or gln.
  MISC_FEATURE (9)..(9): Other Information  Xaa at position 9 is selected from gln, trp, phe, asn, pro, tyr, his, glu, or asp, wherein at least one of Xaa(9), Xaa(10), and Xaa(11) is one of glu, asp, phe, trp, or tyr.
  MISC_FEATURE (10)..(10): Other Information  Xaa at position 10 is selected from glu, asp, trp, tyr, met, asn, pro, gln, or phe, wherein at least one of Xaa(9), Xaa(10), and Xaa(11) is one of glu, asp, phe, trp, or tyr.
  MISC_FEATURE (11)..(11): Other Information  Xaa at position 11 is selected from gln, tyr, phe, trp, glu, val, asn, or asp, wherein at least one of Xaa(9), Xaa(10), and Xaa(11) is one of glu, asp, phe, trp, or tyr.
XXXXXGPXXXX

SEQ ID NO: 7 - protein, 16 aa, Homo sapiens
  MISC_FEATURE (6)..(6): Other Information  Xaa at position 6 is selected from arg, lys, his, gln, asn, glu, asp, thr, ser, trp, tyr, phe, met, ile, leu, val, ala, gly, or pro.
MEPLGXQLTSGPNQEV

SEQ ID NO: 8 - protein, 13 aa, Homo sapiens
  MISC_FEATURE (5)..(5): Other Information  Xaa at position 5 is selected from arg, lys, his, gln, asn, glu, asp, trh, ser, trp, tyr, phe, met, ile, leu, val, ala, gly, or pro.
  MISC_FEATURE (6)..(6): Other Information  Xaa at position 6 is selected from arg, lys, his, gln, asn, glu, asp, trh, ser, trp, tyr, phe, met, ile, leu, val, ala, gly, or pro.
  MISC_FEATURE (7)..(7): Other Information  Xaa at position 7 is selected from arg, lys, his, gln, asn, glu, asp, trh, ser, trp, tyr, phe, met, ile, leu, val, ala, gly, or pro.
  MISC_FEATURE (8)..(8): Other Information  Xaa at position 8 is selected from arg, lys, his, gln, asn, glu, asp, trh, ser, trp, tyr, phe, met, ile, leu, val, ala, gly, or pro.
FRQLXXXXNQEQV

SEQ ID NO: 9 - protein, 8 aa, Homo sapiens
  MISC_FEATURE (1)..(1): Xaa at position 1 is selected from arg, his, lys, thr, ser, trp, gly, ala, gln, asn, ile, leu, met, phe, val, pro, or tyr, wherein at least one of Xaa(1), Xaa(2), and Xaa(3) is arg, his, or lys, and wherein one or two of Xaa(1), Xaa(2), Xaa(3) may be absent.
  MISC_FEATURE (2)..(2): Xaa at position 2 is selected from arg, his, lys, thr, ser, trp, gly, ala, gln, asn, ile, leu, met, phe, val, pro, or tyr, wherein at least one of Xaa(1), Xaa(2), and Xaa(3) is arg, his, or lys, and wherein one or two of Xaa(1), Xaa(2), Xaa(3) may be absent.
  MISC_FEATURE (3)..(3): Xaa at position 3 is selected from arg, his, lys, thr, ser, trp, gly, ala, gln, asn, ile, leu, met, phe, val, pro, or tyr, wherein at least one of Xaa(1), Xaa(2), and Xaa(3) is arg, his, or lys, and wherein one or two of Xaa(1), Xaa(2), Xaa(3) may be absent.
  MISC_FEATURE (4)..(4): Other Information  Xaa at position 4 is selected from thr, ser, trp, gly, ala, gln, ile, leu, met, phe, val, pro, tyr, asn, or his.
  MISC_FEATURE (5)..(5): Other Information Xaa at position 5 is selected from thr, ser, trp, gly, ala, gln, ile, leu, met, phe, val, pro, tyr, asn, or his.
  MISC_FEATURE (8)..(8): Other Information  Xaa at position 8 is selected from asn, ser, his, tyr, ala, phe, or gln.
XXXXXGPX

SEQ ID NO: 10 - protein, 5 aa, Artificial sequence
  Completely synthesized.
GPNQE

SEQ ID NO: 11 - protein, 13 aa, Artificial sequence
  Completely synthesized.
  MISC_FEATURE (8)..(8): Other Information  Xaa at position 8 is pipecolic acid.
FRQLYSGXNQEQV

SEQ ID NO: 12 - protein, 7 aa, Artificial sequence
  Completely synthesized.
  MISC_FEATURE (2)..(2): Other Information  Xaa at position 2 is pipecolic acid.
GXNQEQV